(19) United States
(12) Patent Application Publication
(10) Pub. No.: US 2023/0326613 A1
(43) Pub. Date: Oct. 12, 2023

(54) INFORMATION PROCESSING APPARATUS, INFORMATION PROCESSING METHOD, AND STORAGE MEDIUM

(71) Applicant: NEC Corporation, Minato-ku, Tokyo (JP)

(72) Inventor: Ritsuko Nakahira, Tokyo (JP)

(73) Assignee: NEC Corporation, Minato-ku, Tokyo (JP)

(21) Appl. No.: 18/025,124

(22) PCT Filed: Sep. 9, 2020
(86) PCT No.: PCT/JP2020/034069

§ 371 Date: Mar. 7, 2023

(51) Int. Cl. G16H 50/80; G16H 10/60; G06V 40/16

(52) U.S. Cl. CPC G16H 50/80; G16H 10/60; G06V 40/161

(57) ABSTRACT

An information processing apparatus according to the present invention includes: an acquisition unit that acquires health information about medical treatment performed at a medical institution of a first country on a user scheduled to travel from the first country to a second country; and a request unit that, before the user performs a procedure related to departure, requests quarantine screening on the user for entry to the second country based on the health information.

### TECHNICAL FIELD

The present invention relates to an information processing apparatus, an information processing method, and a storage medium.

### BACKGROUND ART

Patent Literature 1 discloses an infection information evaluation system that detects a physical quantity such as a body temperature or a virus concentration indicating the health condition of a user (passenger) in a security inspection site in an airport and thereby generates infection information indicating the possibility that the user is infected with a contagious disease.

### CITATION LIST

### Patent Literature

PTL 1: Japanese Patent Application Laid-Open No. 2019-204349

### SUMMARY OF INVENTION

### Technical Problem

In recent years, in terms of preventing the spread of infection of contagious diseases in an airport, there is a demand for shortening the time a user spends at the airport from his/her arrival to boarding on an airplane. When health conditions of users are inspected in the airport as with the system described in Patent Literature 1, however, the time a user spends at the airport may be further extended.

Accordingly, in view of such circumstances, the present invention intends to provide an information processing apparatus, an information processing method, and a storage medium that can check the health condition of a user while shortening the time the user spends at an airport.

### Solution to Problem

According to one aspect of the present invention, provided is an information processing apparatus including: an acquisition unit that acquires health information about medical treatment performed at a medical institution of a first country on a user scheduled to travel from the first country to a second country; and a request unit that, before the user performs a procedure related to departure, requests quarantine screening on the user for entry to the second country based on the health information.

According to another aspect of the present invention, provided is an information processing method including: acquiring health information about medical treatment performed at a medical institution of a first country on a user scheduled to travel from the first country to a second country; and before the user performs a procedure related to departure, requesting quarantine screening on the user for entry to the second country based on the health information.

According to yet another aspect of the present invention, provided is a storage medium storing a program that causes a computer to perform: acquiring health information about medical treatment performed at a medical institution of a first country on a user scheduled to travel from the first country to a second country; and before the user performs a procedure related to departure, requesting quarantine screening on the user for entry to the second country based on the health information.

According to yet another aspect of the present invention, provided is an information processing apparatus including: an acquisition unit that acquires health information about medical treatment performed at a medical institution of a first country on a user scheduled to travel from the first country to a second country; and a screening unit that, before the user performs a procedure related to departure, performs quarantine screening on the user for entry to the second country based on the health information.

According to yet another aspect of the present invention, provided is an information processing method including: acquiring health information about medical treatment performed at a medical institution of a first country on a user scheduled to travel from the first country to a second country; and before the user performs a procedure related to departure, performing quarantine screening on the user for entry to the second country based on the health information.

According to yet another aspect of the present invention, provided is a storage medium storing a program that causes a computer to perform: acquiring health information about medical treatment performed at a medical institution of a first country on a user scheduled to travel from the first country to a second country; and before the user performs a procedure related to departure, performing quarantine screening on the user for entry to the second country based on the health information.

### Advantageous Effects of Invention

According to the present invention, it is possible to provide an information processing apparatus, an information processing method, and a storage medium that can check the health condition of a user while shortening the time the user spends at an airport.

### BRIEF DESCRIPTION OF DRAWINGS

FIG. 1 is a block diagram illustrating an example of an overall configuration of an information processing system according to a first example embodiment.

FIG. 2 is a diagram illustrating an example of information stored in a quarantine screening information DB according to the first example embodiment.

FIG. 3 is a diagram illustrating an example of information stored in a token ID information DB according to the first example embodiment.

FIG. 4 is a diagram illustrating an example of information stored in a passage history information DB according to the first example embodiment.

FIG. 5 is a diagram illustrating an example of information stored in an operation information DB according to the first example embodiment.

FIG. 6 is a block diagram illustrating an example of a hardware configuration of a user terminal according to the first example embodiment.

FIG. 7 is a block diagram illustrating an example of a hardware configuration of a screening server according to the first example embodiment.

FIG. 8 is a block diagram illustrating an example of a hardware configuration of a check-in terminal according to the first example embodiment.

FIG. 9 is a block diagram illustrating an example of a hardware configuration of an automatic baggage drop-off machine according to the first example embodiment.

FIG. 10 is a block diagram illustrating an example of a hardware configuration of a security inspection apparatus according to the first example embodiment.

FIG. 11 is a block diagram illustrating an example of a hardware configuration of a second automated gate apparatus according to the first example embodiment.

FIG. 12 is a block diagram illustrating an example of a hardware configuration of a signage terminal according to the first example embodiment.

FIG. 13A is a sequence chart illustrating an example of a process in a quarantine screening procedure of the information processing system according to the first example embodiment.

FIG. 13B is a sequence chart illustrating an example of a process in a quarantine screening procedure of the information processing system according to the first example embodiment.

FIG. 14 is a diagram illustrating an example of an account creation window displayed on the user terminal according to the first example embodiment.

FIG. 15 is a diagram illustrating an example of a travel information entry window displayed on the user terminal according to the first example embodiment.

FIG. 16 is a diagram illustrating an example of a health information entry window displayed on the user terminal according to the first example embodiment.

FIG. 17 is a diagram illustrating an example of a screening result display window displayed on the user terminal according to the first example embodiment.

FIG. 18 is a sequence chart illustrating an example of a process in a check-in procedure of the information processing system according to the first example embodiment.

FIG. 19 is a sequence chart illustrating an example of a process in a security inspection procedure of the information processing system according to the first example embodiment.

FIG. 20A is a sequence chart illustrating an example of a guidance process in the second country of the information processing system according to the first example embodiment.

FIG. 20B is a sequence chart illustrating an example of a guidance process in the second country of the information processing system according to the first example embodiment.

FIG. 21 is a diagram illustrating an example of a guidance window displayed on the signage terminal according to the first example embodiment.

FIG. 22A is a sequence chart illustrating an example of a process in a quarantine screening procedure of the information processing system according to the first example embodiment.

FIG. 22B is a sequence chart illustrating an example of a process in a quarantine screening procedure of the information processing system according to the first example embodiment.

FIG. 23A is a sequence chart illustrating an example of a process in a quarantine screening procedure of the information processing system according to the first example embodiment.

FIG. 23B is a sequence chart illustrating an example of a process in a quarantine screening procedure of the information processing system according to the first example embodiment.

FIG. 24 is a block diagram illustrating an example of an overall configuration of a screening server according to a second example embodiment.

FIG. 25 is a diagram illustrating an example of information stored in an entry history information DB according to the second example embodiment.

FIG. 26 is a diagram illustrating an example of information stored in a marked person information DB according to the second example embodiment.

FIG. 27A is a sequence chart illustrating an example of a process of a pre-immigration inspection procedure of an information processing system according to the second example embodiment.

FIG. 27B is a sequence chart illustrating an example of a process of a pre-immigration inspection procedure of the information processing system according to the second example embodiment.

FIG. 28 is a sequence chart illustrating an example of a process in a check-in procedure of an information processing system according to a third example embodiment.

FIG. 29 is a function block diagram of an information processing apparatus according to a fourth example embodiment.

FIG. 30 is a function block diagram of an information processing apparatus according to a fifth example embodiment.

### DESCRIPTION OF EMBODIMENTS

Exemplary example embodiments of the present invention will be described below with reference to the drawings. Throughout the drawings, similar or corresponding elements are labeled with the same references, and the description thereof may be omitted or simplified.

### First Example Embodiment

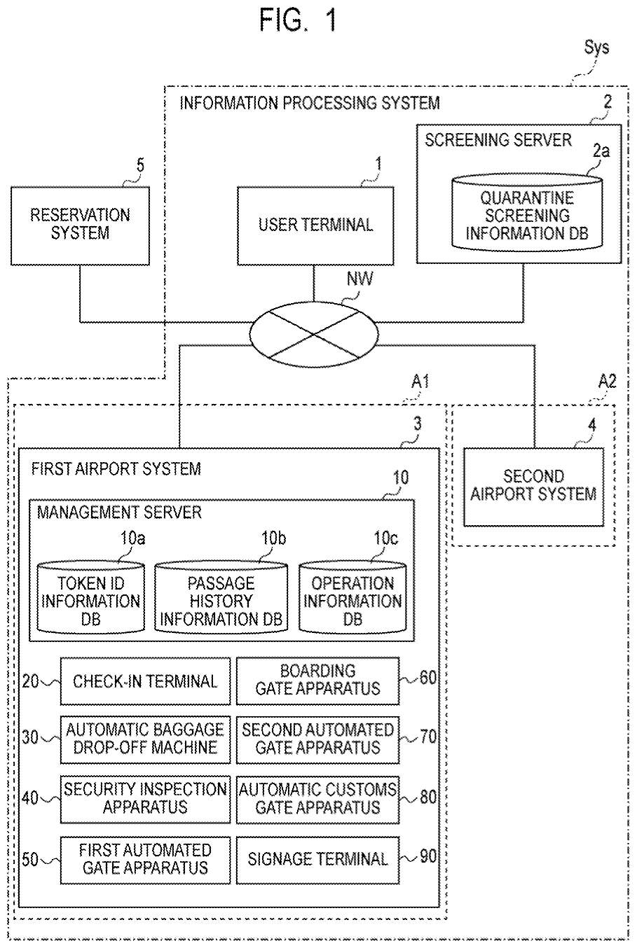

FIG. 1 is a block diagram illustrating an example of the overall configuration of an information processing system Sys according to the present example embodiment. The information processing system Sys according to the present example embodiment is a computer system that supports a series of procedures performed on a user in a first country and a second country, respectively, when the user departs from the first country at an airport A1 of the first country and enters the second country at an airport A2 of the second country by an airplane, for example. The information processing system Sys is run by a public institution such as an immigration control bureau or a trustee entrusted with the operation from such an institution, for example.

As illustrated in FIG. 1, the information processing system Sys includes a user terminal 1, an screening server 2, a first airport system 3, and a second airport system 4. The user terminal 1, the screening server 2, the first airport system 3, and the second airport system 4 are connected to each other via a network NW. The network NW is formed of a Wide Area Network (WAN) such as the Internet. The connection scheme may be a wireless scheme without being limited to a wired scheme. Note that, although only two airport systems are illustrated in FIG. 1 for simplified illustration, the number of airport systems included in the information processing system Sys is not limited thereto.

The user terminal 1 is a terminal device possessed by the user. An example of the user terminal 1 may be, for example, a smartphone, a tablet device, a personal computer, or the like. The user terminal 1 requests the screening server 2 for screening related to entry of the user to the second country. Specifically, once health information about medical treatment performed in a medical institution in the first country is input via a user's input operation, the user terminal 1 requests the screening server 2 for quarantine screening related to the entry to the second country based on the health information.

The screening server 2 is a server installed in a facility of a government agency (public institution) of the second country. The screening server 2 is installed at an immigration control bureau, a health bureau for disease control, or the like, for example. The screening server 2 performs quarantine screening related to the entry of the user to the second country based on health information received from the user terminal 1. The health information includes an inspection category, an inspection date, an inspection result, and the like.

Note that servers of the first airport system 3 and the second airport system 4 may include some or all of the functions of the screening server 2, which are outsourced from a government agency. Further, a server similar to the screening server 2 may be installed in the first country. The screening server 2 is not required to be a single server and may be configured as a server group including a plurality of servers. Further, the screening server 2 is not necessarily required to be provided on a country basis and may be configured as a server used by a plurality of countries in a shared manner.

The first airport system 3 is a computer system installed in the airport (departure airport) A1 of the first country and manages various procedures on the user during entry to and departure from the country. The second airport system 4 is a computer system installed in the airport (arrival airport) A2 of the second country. Further, a reservation system 5 of an airline company is connected to the network NW. The reservation system 5 is a computer system installed in a facility of the airline company and accepts a boarding reservation from the user.

Further, as illustrated in FIG. 1, the screening server 2 includes a quarantine screening information DB 2a. This database is an example, and the screening server 2 may further include another database. Further, a plurality of databases may be aggregated into a single database.

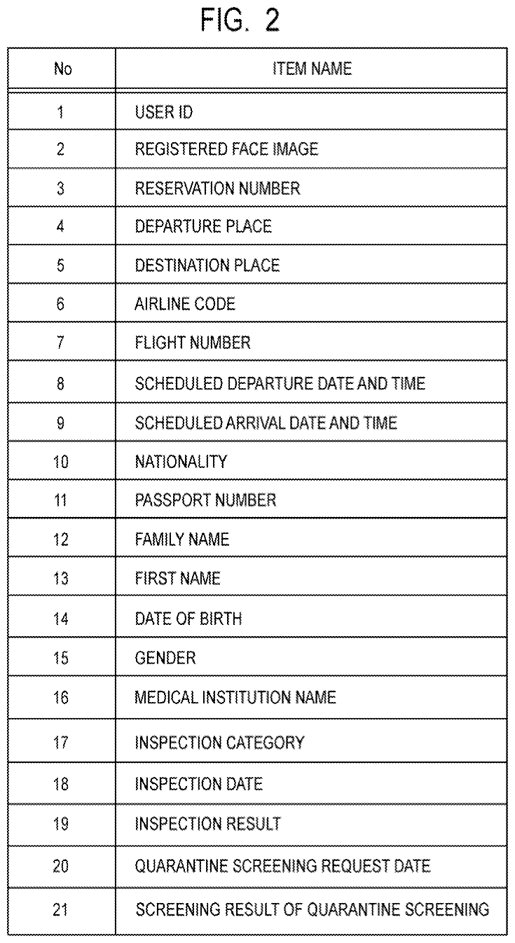

The quarantine screening information DB 2a is a database that stores information on quarantine screening for users who are scheduled to enter the second country and entrants who have entered the second country. The quarantine screening of the present example embodiment is screening for determining whether or not the health condition of the user satisfies a requirement related to quarantine defined by a law or a rule in the second country. FIG. 2 is a diagram illustrating an example of information stored in the quarantine screening information DB 2a. The quarantine screening information DB 2a has data items of a user ID, a registered face image, a reservation number, a departure place, a destination place, an airline code, a flight number, a scheduled departure date and time, a scheduled arrival date and time, a nationality, a passport number, a family name, a first name, a date of birth, a gender, a medical institution name, an inspection category, an inspection date, an inspection result, a quarantine screening request date, and a screening result of a quarantine screening.

The user ID is identification information that uniquely identifies a user who has requested quarantine screening. The registered face image is a face image registered for a user. As a registered face image stored in the quarantine screening information DB 2a, a captured face image or a passport face image received from the user terminal 1 is used. The reservation number is an identifier that uniquely identifies boarding reservation information. The airline code is an identifier that uniquely identifies an airline company. The medical institution name is a name or an identification code of a medical institution where the user was inspected in the first country. The inspection category indicates a type of inspection. The inspection date is a date or a date and time when the inspection was performed in a medical institution. The quarantine screening request date is a date or a date and time when the user requested the screening server 2 for quarantine screening from the user terminal 1.

Further, as illustrated in FIG. 1, the first airport system 3 includes a management server 10, a check-in terminal 20, an automatic baggage drop-off machine 30, a security inspection apparatus 40, a first automated gate apparatus 50, a boarding gate apparatus 60, and a second automated gate apparatus 70, an automatic customs gate apparatus 80, and a signage terminal 90. The same applies to the second airport system 4. The management server 10 is connected to other apparatuses via a network (not illustrated) such as a LAN, respectively. The connection scheme may be a wireless scheme without being limited to a wired scheme.

The management server 10 is an information processing apparatus that manages various procedures on the user during entry to or departure from a country. The management server 10 is installed in a facility of an airport company, an airline company, or the like, for example. Note that the management server 10 is not required to be a single server and may be configured as a server group including a plurality of servers. Further, the management server 10 is not necessarily required to be provided on a country basis and may be configured as a server used by a plurality of countries in a shared manner.

The management server 10 performs identity verification on a user by matching a face image captured by the check-in terminal 20, which is a face authentication terminal, with a passport face image read from a passport by the check-in terminal 20.

Furthermore, the management server 10 performs identity verification on a user by matching a face image captured by another face authentication terminal (each of the automatic baggage drop-off machine 30, the security inspection apparatus 40, the first automated gate apparatus 50, the boarding gate apparatus 60, the second automated gate apparatus 70, the automatic customs gate apparatus 80, and the signage terminal 90) with a registered face image registered in a database, respectively.

Further, as illustrated in FIG. 1, the management server 10 includes a token ID information DB 10a, a passage history information DB 10b, and an operation information DB 10c. These databases are examples, and the management server 10 may further include other databases. Further, a plurality of databases may be aggregated into a single database.

Further, in the present example embodiment, the databases in the first airport system 3 and the databases in the second airport system 4 are provided separately, and a part or the whole of stored information is synchronized at a suitable timing. For example, it is preferable that information associated with a token ID be also registered to the database on the second airport system 4 side at a timing that the user departs from the first country. Note that only one of the first airport system 3 and the second airport system 4 may include the token ID information DB 10a, the passage history information DB 10b, and the operation information DB 10c, which may be configured to be used in both the first country and the second country.

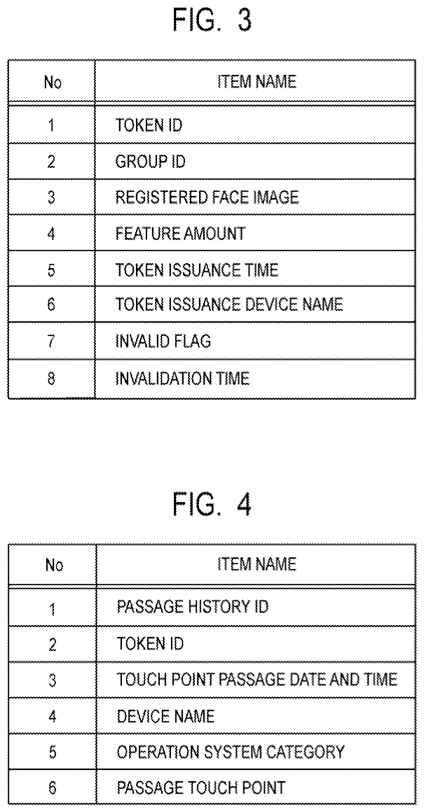

FIG. 3 is a diagram illustrating an example of information stored in the token ID information DB 10a. The token ID information DB 10a has data items of a token ID, a group ID, a registered face image, a feature amount, a token issuance time, a token issuance device name, an invalid flag, and an invalidation time.

The token ID is an identifier that uniquely identifies ID information. The token ID of the present example embodiment is issued by the management server 10 provided that a result of a matching process is that the matching is successful where the matching process is to match a captured face image, which is obtained by the user capturing by himself/herself by using a face authentication terminal such as the check-in terminal 20, with a passport face image read from a passport by the face authentication terminal. Further, for example, after the user finishes the travel from the first country to the second country, the token ID is invalidated. That is, a token ID is not an identifier used permanently but a onetime ID having a validity period (lifecycle).

Note that the term “matching is successful” in the present example embodiment means that a matching score indicating a similarity between biometric information on a user and registered biometric information on a registrant is greater than or equal to a predetermined threshold. In contrast, the term “matching is unsuccessful” means that a matching score is less than the predetermined threshold.

The group ID is an identifier for grouping ID information. The registered face image is a face image registered for a user. In the present example embodiment, a face image captured during a procedure in the airport A1 of the first country or a passport face image read from an IC chip of a passport by a reading device is used as a registered face image stored in the token ID information DB 10a. The feature amount is a value extracted from biometric information (registered face image).

Note that, although the term of biometric information in the present example embodiment means a face image and a feature amount extracted from the face image, the biometric information is not limited to a face image and a face feature amount. That is, biometric authentication may be performed by using an iris image, a fingerprint image, a palmprint image, an auricular image, or the like as user biometric information.

The token issuance time is a time that the management server 10 issued a token ID. The token issuance device name is a device name from which a registered face image which triggered issuance of a token ID is acquired. The invalid flag is flag information indicating whether or not a token ID is currently valid. For example, upon issuance of a token ID, the invalid flag is set to a value indicating a state where the token ID is valid. Further, in response to satisfying a predetermined condition, the invalid flag is updated to a value indicating a state where a token ID is invalid. The invalidation time is a timestamp indicating a time the invalid flag is invalidated.

FIG. 4 is a diagram illustrating an example of information stored in the passage history information DB 10b. The passage history information DB 10b has data items of a passage history ID, a token ID, a touch point passage date and time, a device name, an operation system category, and a passage touch point. The passage history ID is an identifier that uniquely identifies passage history information. The touch point passage date and time is a timestamp indicating a time the user passes through a touch point. The device name is a machine name of an operation terminal used for a procedure at a touch point. The operation system category is a category of an operation system which an operation terminal belongs to. The passage touch point is a name of a touch point through which the user passes. Note that, by extracting passage history information on a token ID basis, the management server 10 can know up to which touch point the user has completed the procedure.

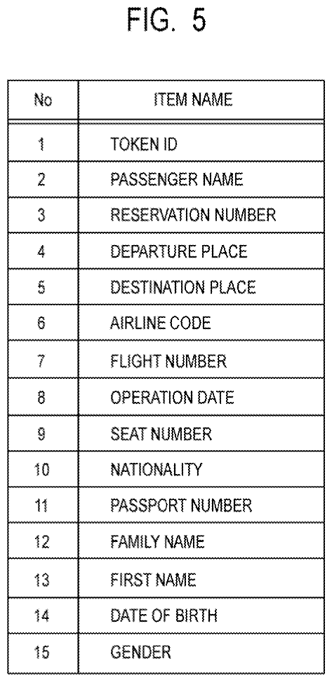

FIG. 5 is a diagram illustrating an example of information stored in the operation information DB 10c. The operation information DB 10c has data items of a token ID, a passenger name, a reservation number, a departure place, a destination place, an airline code, a flight number, an operation date, a seat number, a nationality, a passport number, a family name, a first name, a date of birth, and a gender. In such a way, the operation information DB 10c stores operation information about a predetermined operation in association with a token ID. In the present example embodiment, the “predetermined operation” means a procedural operation (check-in/baggage drop-off/security inspection/departure inspection/passenger identity verification/entry inspection/customs inspection) performed at each touch point in an airport.

The operation information includes boarding reservation information such as a passenger name, a reservation number, a departure place, a destination place, an airline code, a flight number, an operation date, a seat number, a nationality, a passport number, a family name, a first name, a date of birth, and a gender.

Next, the apparatuses responsible for procedural operations on users in cooperation with the management server 10 in the first airport system 3 and the second airport system 4 will be described.

The check-in terminal 20 is installed in a check-in lobby or a check-in counter in each of the airport A1 and the airport A2. Hereafter, the procedural area where the check-in terminal 20 is installed is referred to as “touch point TP1”. The check-in terminal 20 is a self-service terminal operated by the user by himself/herself to perform a check-in procedure (a boarding procedure). After completion of the check-in procedure at the touch point TP1, the user moves to a baggage drop-off place or a security inspection site.

The automatic baggage drop-off machine 30 is installed in a region adjacent to a baggage counter (a manned counter) or a region near the check-in terminal 20 in each of the airport A1 and the airport A2. Hereafter, the procedural area where the automatic baggage drop-off machine 30 is installed is referred to as “touch point TP2”. The automatic baggage drop-off machine 30 is a self-service terminal operated by the user by himself/herself to perform a procedure to drop off, to an airline company, baggage not to be carried in the cabin. After completion of the baggage drop-off procedure at the touch point TP2, the user moves to the security inspection site. When the user does not drop off his/her baggage, the procedure at the touch point TP2 is omitted.

The security inspection apparatus 40 is installed in the security inspection site (hereafter, referred to as “touch point TP3”) in each of the airport A1 and the airport A2. The term “security inspection apparatus” in the present example embodiment is used as a meaning including all of a metal detector that checks whether or not the user is wearing a metal item that may be a dangerous object, an X-ray inspection device that uses an X-ray to check whether or not a dangerous object is included in carry-on baggage or the like, a passage control device that determines whether or not to permit passage of the user at an entrance or an exit of the security inspection site, and the like. After completion of the security inspection procedure at the touch point TP3, the user moves to a departure inspection site.

The first automated gate apparatus 50 is installed at the departure inspection site (hereafter, referred to as “touch point TP4”) in each of the airport A1 and the airport A2. The first automated gate apparatus 50 is an apparatus that automatically performs a departure inspection procedure on the user. After completion of the departure inspection procedure at the touch point TP4, the user moves to a departure area where a duty-free shop and a boarding gate are provided.

The boarding gate apparatus 60 is installed to each boarding gate (hereafter, referred to as “touch point TP5”) in each of the airport A1 and the airport A2. The boarding gate apparatus 60 is a passage control apparatus that checks whether or not the user is a passenger of an airplane associated with the boarding gate. After completion of the procedure at the touch point TP5, the user boards the airplane and departs to the second country. In such a way, the check-in terminal 20, the automatic baggage drop-off machine 30, the security inspection apparatus 40, the first automated gate apparatus 50, and the boarding gate apparatus 60 are used when the user departs from the first country.

Further, the first automated gate apparatus 50 is installed in each entry inspection site (hereafter, referred to as “touch point TP6”) in each of the airport A1 and the airport A2 and automatically performs an entry inspection procedure on the user. In the present example embodiment, after completion of the entry inspection procedure at the touch point TP6, the user moves to a quarantine station.

The second automated gate apparatus 70 is installed in each quarantine station (hereafter, referred to as “touch point TP7”) in each of the airport A1 and the airport A2. The second automated gate apparatus 70 opens gate when a screening result of quarantine screening performed in advance on the user is no problem (“permit”). Then, after passing through the second automated gate apparatus 70, the user moves to a baggage claim area or a customs inspection site. Note that the order of performing entry inspection and quarantine screening is not limited to the above. That is, the quarantine screening may be performed before the entry inspection.

The automatic customs gate apparatus 80 is installed in each customs inspection site (hereafter, referred to as “touch point TP8”) in each of the airport A1 and the airport A2. The automatic customs gate apparatus 80 is an apparatus that automatically performs a customs inspection procedure on the user. After completion of the procedure at the touch point TP8, the user is able to enter the second country. In such a way, the first automated gate apparatus 50, the second automated gate apparatus 70, and the automatic customs gate apparatus 80 are used when the user enters the second country.

The signage terminal 90 is installed in any place of each of the airport A1 and the airport A2. The signage terminal 90 is a display terminal for presenting, to the user, various guidance information received from the management server 10. The signage terminal 90 of the present example embodiment is at least installed near the entrance of the quarantine station (touch point TP7). After performing biometric authentication based on user biometric information (captured face image), the management server 10 inquires the screening server 2 for a screening result of quarantine screening and thereby determines whether or not the user is a person who is allowed to use the second automated gate apparatus 70 or whether or not the user is a subject of face-to-face quarantine inspection. The signage terminal 90 displays guidance information about quarantine screening to the user based on a determination result at the management server 10.

Next, a hardware configuration of devices forming the information processing system Sys will be described. Note that, throughout a plurality of drawings, devices having the same name and differing only in the reference are devices having substantially the same function, and thus, the detailed description thereof will be omitted in the subsequent drawings.

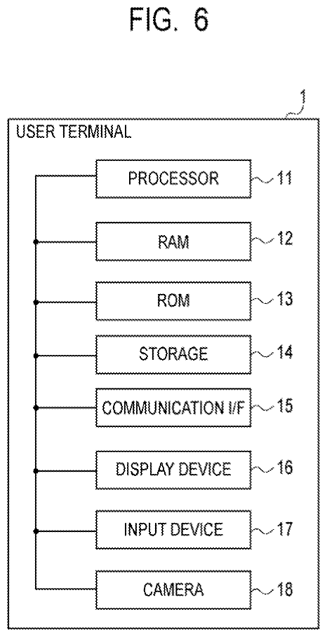

FIG. 6 is a block diagram illustrating an example of a hardware configuration of the user terminal 1. The user terminal 1 includes a processor 11, a random access memory (RAM) 12, a read only memory (ROM) 13, a storage 14, a communication interface (I/F) 15, a display device 16, an input device 17, and a camera 18, as a computer that performs calculation, control, and storage. These devices are connected to each other via a bus, a wiring, a drive device, or the like.

The processor 11 has functions of performing predetermined calculation in accordance with a program stored in the ROM 13, the storage 14, or the like and controlling each unit of the user terminal 1. Further, a central processing unit (CPU), a graphics processing unit (GPU), a field programmable gate array (FPGA), or the like may be used as the processor 11.

The RAM 12 is formed of a volatile storage medium and provides a temporary memory area required for the operation of the processor 11. The ROM 13 is formed of a nonvolatile storage medium and stores information required such as a program used for the operation of the user terminal 1.

The storage 14 is formed of a nonvolatile storage medium and performs storage of a database, storage of a operating program of the user terminal 1, or the like. The storage 14 is formed of a hard disk drive (HDD) or a solid state drive (SSD), for example.

The communication I/F 15 is a communication interface based on a specification such as Ethernet (registered trademark), Wi-Fi (registered trademark), 4G, or the like and is a module for communicating with other devices.

The display device 16 is a liquid crystal display, an organic light emitting diode (OLED) display, or the like configured to display a moving image, a static image, a text, or the like and is used for presenting information to the user.

The input device 17 is a keyboard, a pointing device, a button, or the like and accepts a user operation. The display device 16 and the input device 17 may be formed integrally as a touch panel.

The camera 18 is a digital camera having a Complementary Metal-Oxide-Semiconductor (CMOS) image sensor, a Charge Coupled Device (CCD) image sensor, or the like as a light receiving device, for example. The camera 18 of the present example embodiment is used for the user to capture an image of his/her face and an image of a face photograph attached to his/her passport.

The processor 11 loads a program stored in the ROM 13, the storage 14, or the like into the RAM 12 and executes the program to perform a predetermined calculation process. Further, the processor 11 controls each unit of the user terminal 1, such as the communication I/F 15, based on the program.

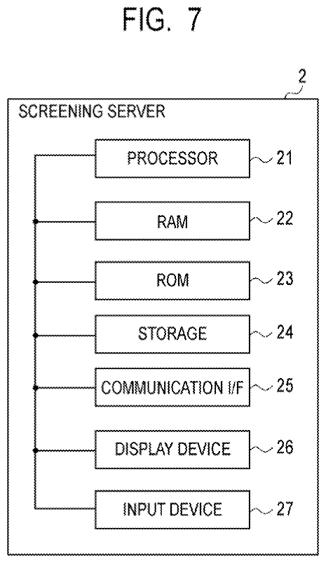

FIG. 7 is a block diagram illustrating an example of the hardware configuration of the screening server 2. The screening server 2 includes a processor 21, a RAM 22, a ROM 23, a storage 24, a communication I/F 25, a display device 26, and an input device 27. These devices are connected to each other via a bus, a wiring, a drive device, or the like. Further, as the processor 21, one of a CPU, a GPU, a FPGA, a digital signal processor (DSP), and an application specific integrated circuit (ASIC) may be used, or a plurality thereof may be used in parallel. Note that, since the hardware configuration of the management server 10 is the same as that of the screening server 2, the description thereof will be omitted.

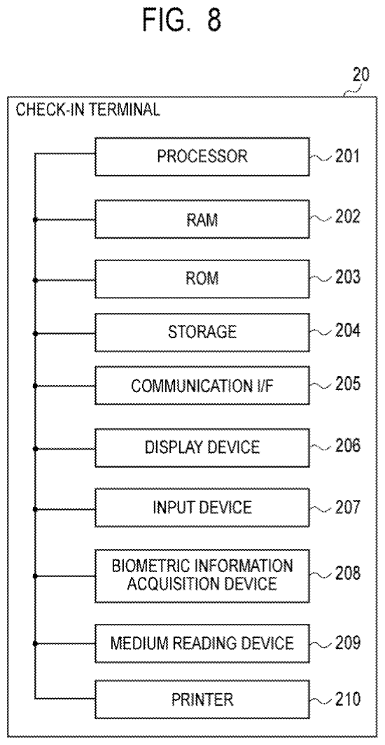

FIG. 8 is a block diagram illustrating an example of the hardware configuration of the check-in terminal 20. The check-in terminal 20 includes a processor 201, a RAM 202, a ROM 203, a storage 204, a communication I/F 205, a display device 206, an input device 207, a biometric information acquisition device 208, a medium reading device 209, and a printer 210. These devices are connected to each other via a bus, a wiring, a drive device, or the like.

The biometric information acquisition device 208 is a device that acquires a user face image as user biometric information. The biometric information acquisition device 208 is a digital camera having a CMOS image sensor, a CCD image sensor, or the like as a light receiving element, for example. The biometric information acquisition device 208 captures an image of a face of the user standing in front of the device to acquire the face image, for example.

The medium reading device 209 is a device that reads information recorded or stored in a medium carried by the user. The medium reading device 209 may be, for example, a code reader, an image scanner, a contactless integrated circuit (IC) reader, an optical character reader (OCR) device, or the like. Further, a recording medium or a storage medium may be, for example, a paper airline ticket, a mobile terminal displaying a receipt of an e-ticket, or the like. The printer 210 prints a boarding ticket in which boarding information and guidance information about procedures up to boarding are printed at the time of completion of a check-in procedure.

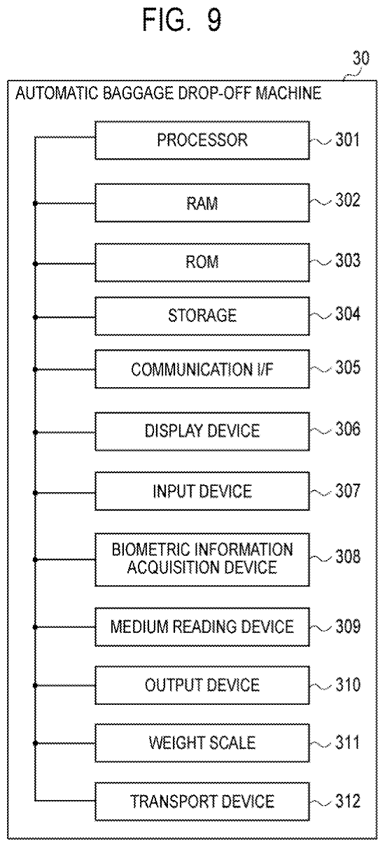

FIG. 9 is a block diagram illustrating an example of the hardware configuration of the automatic baggage drop-off machine 30. The automatic baggage drop-off machine 30 includes a processor 301, a RAM 302, a ROM 303, a storage 304, a communication I/F 305, a display device 306, an input device 307, a biometric information acquisition device 308, a medium reading device 309, an output device 310, a weight scale 311, and a transport device 312. These devices are connected to each other via a bus, a wiring, a drive device, or the like.

The output device 310 is a device that outputs a baggage tag attached to checked baggage. For example, the baggage tag is an RFID tag having an IC chip that stores tag information including a checked baggage ID, a user ID, a flight number, or the like. Further, the output device 310 further outputs a baggage claim tag required for claiming checked baggage after arriving at the destination. The baggage tag or the baggage claim tag is associated with at least one of a reservation number, a boarding ticket number, and a token ID, for example.

The weight scale 311 measures the weight of checked baggage and outputs a measured value to the processor 301. When the weight of checked baggage exceeds a predetermined threshold, the processor 301 outputs error information that urges the user to take some action. The transport device 312 transports checked baggage on a receiving area placed by the user.

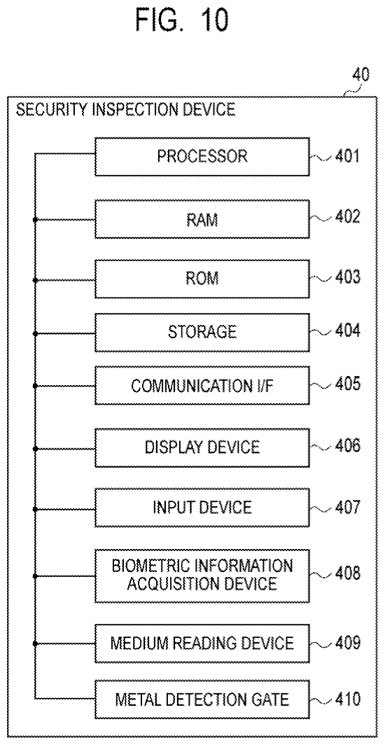

FIG. 10 is a block diagram illustrating an example of the hardware configuration of the security inspection apparatus 40. The security inspection apparatus 40 includes a processor 401, a RAM 402, a ROM 403, a storage 404, a communication I/F 405, a display device 406, an input device 407, a biometric information acquisition device 408, a medium reading device 409, and a metal detection gate 410. These devices are connected to each other via a bus, a wiring, a drive device, or the like.

The metal detection gate 410 is a gate-type metal detector and detects a metal item worn by a user passing through the metal detection gate 410.

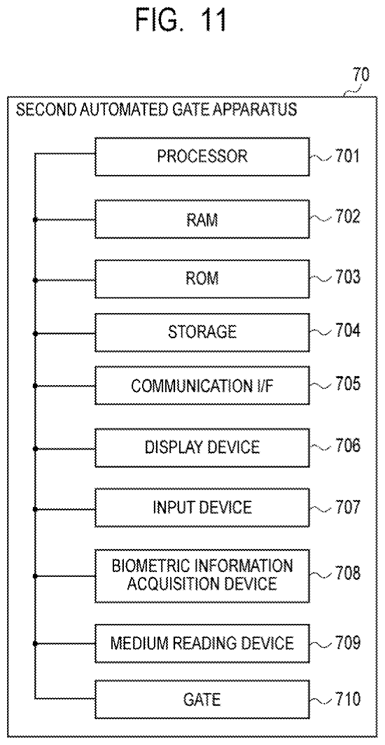

FIG. 11 is a block diagram illustrating an example of the hardware configuration of the second automated gate apparatus 70. The second automated gate apparatus 70 includes a processor 701, a RAM 702, a ROM 703, a storage 704, a communication I/F 705, a display device 706, an input device 707, a biometric information acquisition device 708, a medium reading device 709, and a gate 710. These devices are connected to each other via a bus, a wiring, a drive device, or the like. Note that, since each hardware configuration of the first automated gate apparatus 50, the boarding gate apparatus 60, and the automatic customs gate apparatus 80 is the same as that of the second automated gate apparatus 70, the description thereof will be omitted.

The gate 710 transitions from a closed state to block passage of a user during standby to an open state to permit passage of the user under the control of the processor 701 when identity verification of the user at the second automated gate apparatus 70 is successful. The scheme of the gate 710 is not particularly limited and may be, for example, a flapper gate in which one or more flappers provided to one side or both sides of a passage are opened and closed, a turn style gate in which three bars are revolved, or the like.

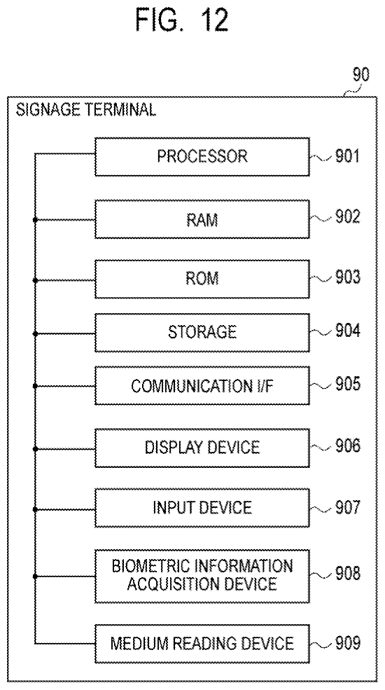

FIG. 12 is a block diagram illustrating an example of the hardware configuration of the signage terminal 90. The signage terminal 90 includes a processor 901, a RAM 902, a ROM 903, a storage 904, a communication I/F 905, a display device 906, an input device 907, a biometric information acquisition device 908, and a medium reading device 909. These devices are connected to each other via a bus, a wiring, a drive device, or the like.

Note that the hardware configurations illustrated in FIG. 6 to FIG. 12 are examples, a device other than the above may be added, and some of the devices may be omitted. Further, some of the devices may be replaced with another device having the same function. Further, some of the functions of the present example embodiment may be provided by another device via a network, or the functions of the present example embodiment may be distributed to and implemented by a plurality of devices. In such a way, the hardware configurations illustrated in FIG. 6 to FIG. 12 can be changed as appropriate.

For example, the user terminal 1 may further include a medium reading device that uses a technology of short-range wireless communication such as Near Field Communication (NFC) to wirelessly communicate with an IC chip embedded in a passport. In such a way, it is possible to read a passport face image or passport information from a passport (IC chip) instead of using the camera 18 to capture an image on a face photograph of a passport.

Next, the operation of the devices in the information processing system Sys according to the present example embodiment will be described with reference to the drawings.

### [Quarantine Screening Procedure]

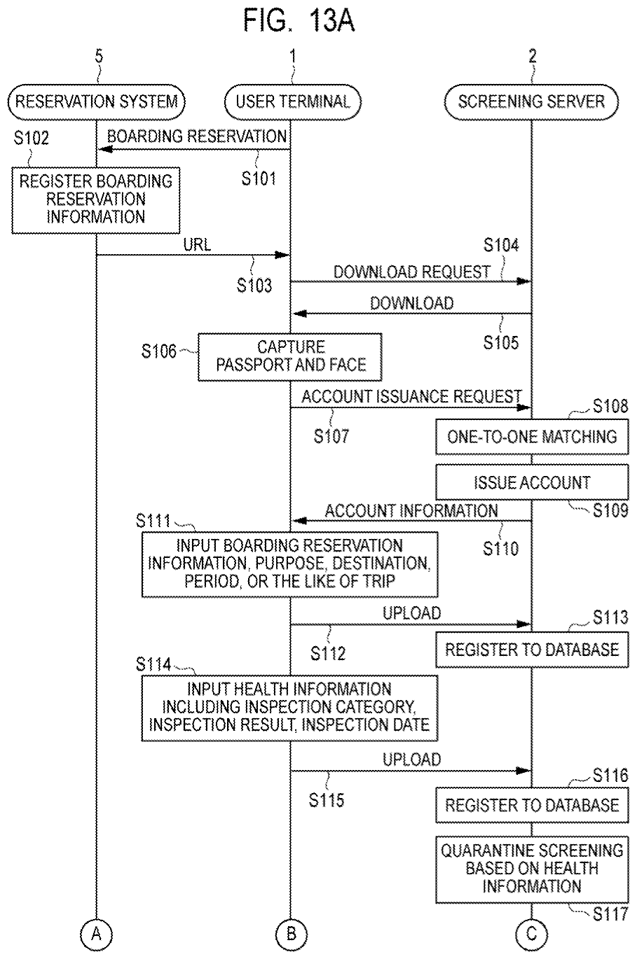

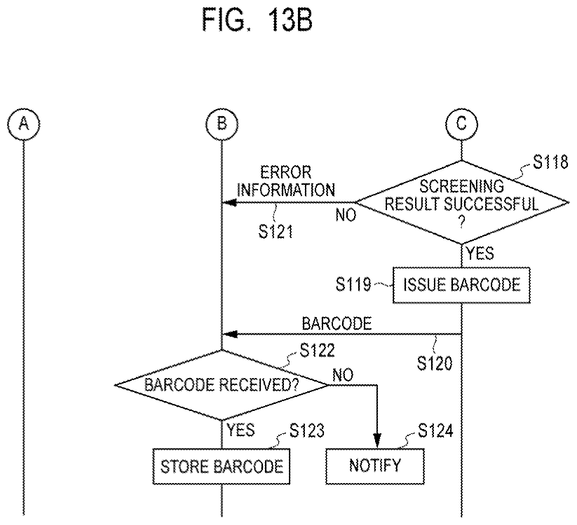

FIG. 13A and FIG. 13B are sequence charts illustrating an example of a process in a quarantine screening procedure of the information processing system Sys according to the present example embodiment.

First, the user terminal 1 accesses the reservation system 5 of an airline company and makes a boarding reservation in accordance with a user operation (step S101).

Next, the reservation system 5 registers boarding reservation information (step S102) and then transmits a URL used for downloading a travel management application to the user terminal 1 (step S103).

Next, the user terminal 1 accesses the screening server 2 based on the URL received from the reservation system 5 and requests download of the application (step S104).

Next, the user terminal 1 downloads the application from the screening server 2 (step S105) and then installs the application in the terminal. Note that, when the application has already been installed in the user terminal 1 since the past travel, the process of step S104 to step S105 can be omitted.

Next, the user terminal 1 captures images of a face photograph attached to a passport and a user face, respectively, in accordance with the user operation (step S106) and then transmits an account issuance request to the screening server 2 (step S107). The data on the account issuance request includes the passport face image and the captured face image.

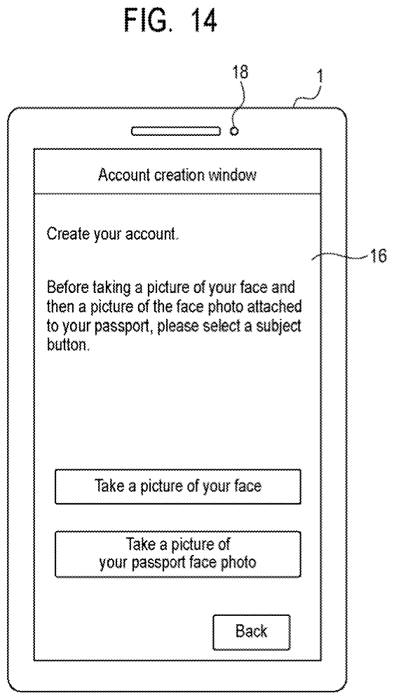

FIG. 14 is a diagram illustrating an example of an account creation window displayed on the user terminal 1. The display device 16 of the user terminal 1 displays a message to guide the user for operations required for account creation (“Create your account. Before taking a picture of your face and then a picture of the face photo attached to your passport, please select a subject button”) and three operation buttons (Take a picture of your face/Take a picture of your passport face photo/Back).

Next, the screening server 2 performs one-to-one matching between the captured face image and the passport face image included in the received data based on the account issuance request received from the user terminal 1 (step S108).

Next, if the matching result of step S108 is that the matching is successful, the screening server 2 issues a user account (step S109). In this step, the screening server 2 registers the issued account information about the user to the quarantine screening information DB 2a.

Next, the screening server 2 transmits the issued account information to the user terminal 1 (step S110). The account information includes a user ID.

Next, the user terminal 1 inputs boarding reservation information, a purpose, a destination, a period, or the like of a trip in an entry form of the window in accordance with a user operation (step S111) and then uploads the input information to the screening server 2 (step S112). Note that the input information is associated with the user ID.

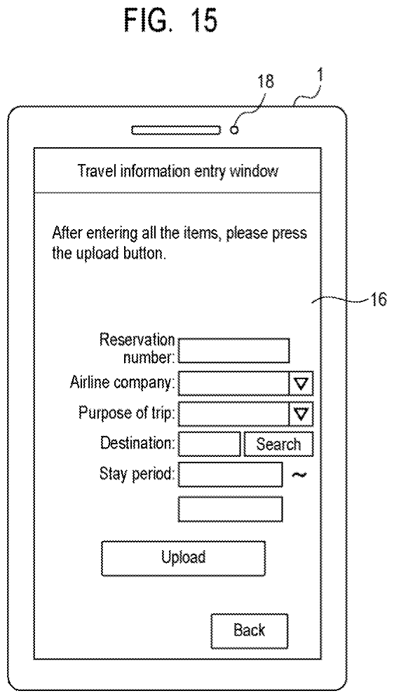

FIG. 15 is a diagram illustrating an example of a travel information entry window displayed on the user terminal 1. The display device 16 of the user terminal 1 displays a message to guide the user about how to operate the window (“After entering all the items, please press the upload button.”), six entry forms (Reservation number/Airline company/Purpose of trip/Destination/Stay period (start date, end date)), and three operation buttons (Search/Upload/Back).

Note that, when the reservation system 5 of an airline company is accessed from the user terminal 1 to make a boarding reservation (step S101 to step S102) as with the present example embodiment, it is preferable that the user terminal 1 cooperate with the reservation system 5 and thereby information be automatically input to the entry form of a reservation number or an airline company of FIG. 15, for example.

Next, the screening server 2 registers the input information received from the user terminal 1 to the quarantine screening information DB 2a by using the user ID as a key (step S113).

Next, the user terminal 1 fills an entry form in the window with health information including an inspection category, an inspection result, an inspection date, or the like in accordance with the user operation (step S114) and then uploads the health information to the screening server 2 (step S115). The inspection may be, for example, a polymerase chain reaction (PCR) test or the like, a genetic test, a blood test, an X-ray test, an ultrasonic test, urinalysis, or the like.

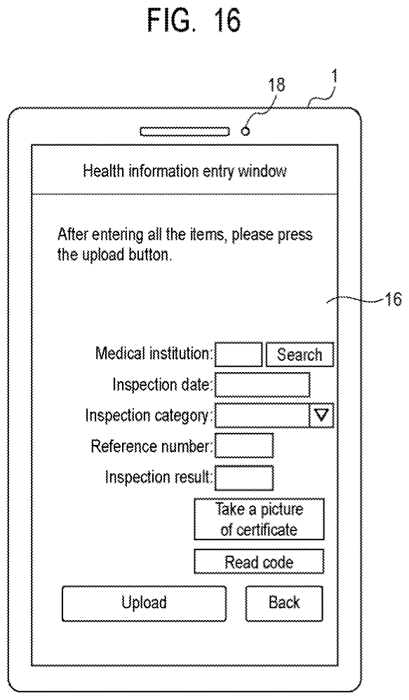

FIG. 16 is a diagram illustrating an example of a health information entry window displayed on the user terminal 1. The display device 16 of the user terminal 1 displays a message to guide the user about how to operate the window (“After entering all the items, please press the upload button.”), five entry forms (Medical institution/Inspection date/Inspection category/Reference number/Inspection result), and five operation buttons (Search/Take picture of certificate/Read code/Upload/Back).

For example, once the button labeled with “Take picture of certificate” is pressed, the camera 18 of the user terminal 1 is started and is ready to capture an image of an inspection certificate of a paper medium provided from a medical institution. Similarly, once the button labeled with “Read code” is pressed, the camera 18 of the user terminal 1 is started and is ready to read a barcode printed in an inspection certificate of a paper medium.

Next, the screening server 2 registers the health information received from the user terminal 1 to the quarantine screening information DB 2a by using the user ID as a key (step S116).

Next, the screening server 2 performs quarantine screening based on the health information received from the user terminal 1 (step S117). In this step, the screening server 2 registers, to the quarantine screening information DB 2a, quarantine screening information in which a screening result of quarantine screening is associated with the user ID.

Next, the screening server 2 determines whether or not the result of the quarantine screening represents permit (step S118). In this step, if the screening server 2 determines that the result of the quarantine screening represents permit (step S118: Yes), the screening server 2 issues a barcode indicating the screening result of “permit” (step S119) and outputs the barcode to the user terminal 1 (step S120). The process then proceeds to step S122.

In contrast, if the screening server 2 determines that the result of the quarantine screening does not represent permit (step S118: NO), the screening server 2 outputs error information to the user terminal 1 (step S121). The process then proceeds to step S122.

Specifically, the screening server 2 outputs the barcode indicating the screening result of “permit” when the inspection date included in the health information is within a predetermined period from the scheduled arrival time at the second country (or the scheduled departure time at the first country) and the inspection result is negative. On the contrary, the screening server 2 outputs the error information indicating the screening result of “not permit” when the above conditions are not satisfied. For example, a case where the predetermined period is 72 hours will be described. When the inspection date that the user was inspected in the first country (inspection date and time) is within 72 hours from the scheduled arrival time at the second country (or the scheduled departure time at the first country), the screening server 2 outputs a screening result of “permit”. On the contrary, when the inspection date (inspection date and time) exceeds 72 hours from the scheduled arrival time at the second country (or the scheduled departure time at the first country), the screening server 2 outputs a screening result of “not permit”.

In step S122, the user terminal 1 determines whether or not the barcode has been received from the screening server 2. In this step, if the user terminal 1 determines that the barcode has been received (step S122: YES), the user terminal 1 stores the barcode, for example, in the storage 14 (step S123) and ends the process.

In contrast, if the user terminal 1 determines that the barcode has not been received from the screening server 2 (step S122: NO), the user terminal 1 notifies the user of an error message (step S124) and ends the process.

Note that the order of performing the face authentication (step S108), the issuance of an account (step S109), the registration of boarding reservation information to the database (step S113), and the quarantine screening based on health information (step S116 to step S118) is not limited to the order illustrated in FIG. 13A and FIG. 13B. These processes may be changed to be performed in any order.

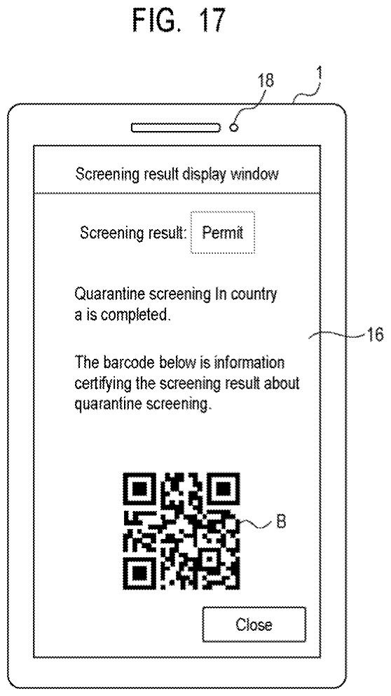

FIG. 17 is a diagram illustrating an example of a screening result display window displayed on the user terminal 1. The display device 16 of the user terminal 1 displays a message to guide the user for a screening result (“Screening result: Permit/Quarantine screening in Country A is completed. The barcode below is information certifying the screening result about quarantine screening.”), a two-dimensional barcode B, and an operation button (Close).

### [Check-In Procedure]

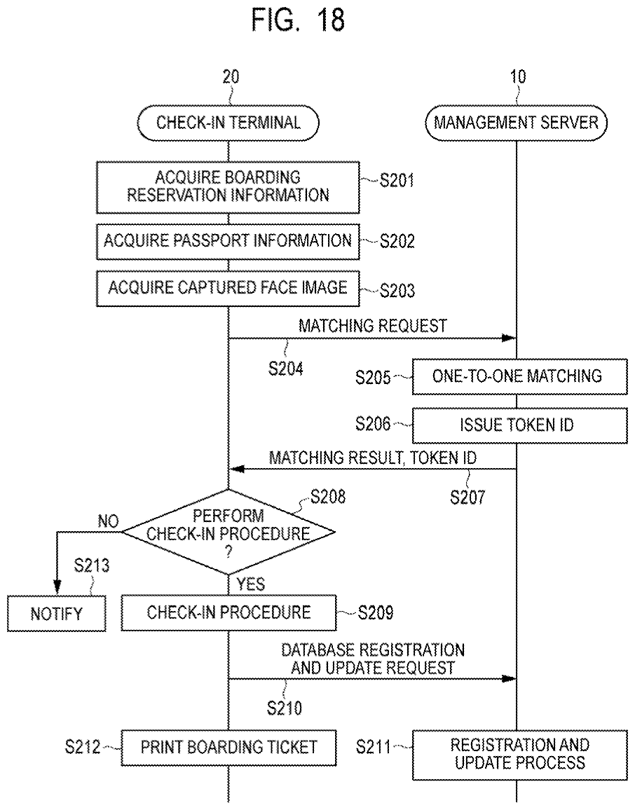

FIG. 18 is a sequence chart illustrating an example of the process during a check-in procedure of the information processing system Sys according to the present example embodiment.

First, in response to an airline ticket medium being held over the reading area of the medium reading device 209, the check-in terminal 20 acquires boarding reservation information on the user from the held airline ticket medium (step S201). The boarding reservation information includes user attribute information (a family name, a first name, a gender, or the like) or flight information (an airline code, a flight number, a boarding date, a departure place (a departure airport), a transit point, a destination place (an arrival airport), a seat number, a departure time, an arrival time, or the like).

Next, when a passport is held over the reading area of the medium reading device 209, the check-in terminal 20 acquires passport information on the user from the held passport (step S202). The passport information includes a passport face image of the user, identity verification information, a passport number, information on a country that has issued the passport, or the like.

Next, the check-in terminal 20 captures an image of the user's face by using the biometric information acquisition device 208 and acquires the captured face image as a target face image (step S203). Note that it is preferable to display a window for obtaining user consent before capturing a face image.

Next, the check-in terminal 20 requests the management server 10 to match face images (step S204). The data of the matching request includes a captured face image captured at the current place and the passport face image read from the passport.

In response to receiving information from the check-in terminal 20, the management server 10 matches at one-to-one the captured face image captured by the check-in terminal 20 with the passport face image (step S205).

The management server 10 issues a token ID provided that the matching result in step S205 is that the matching is successful (step S206) and transmits the matching result and the token ID to the check-in terminal 20 (step S207).

Next, based on the matching result received from the management server 10, the check-in terminal 20 determines whether or not a check-in procedure is ready to be performed (step S208). In this step, if the check-in terminal 20 determines that a check-in procedure is not ready to be performed (step S208: NO), the check-in terminal 20 notifies the user of an error message (step S213) and ends the process.

In contrast, if the check-in terminal 20 determines that a check-in procedure is ready to be performed (step S208: YES), the check-in terminal 20 performs a check-in procedure such as confirmation of an itinerary, selection of a seat, or the like based on user input information (step S209). In response to completion of the check-in procedure, the check-in terminal 20 transmits a database registration and update request to the management server 10 (step S210).

Next, in response to receiving the registration and update request from the check-in terminal 20, the management server 10 performs a registration process and an update process on the passage history information DB 10b and the operation information DB 10c (step S211).

The check-in terminal 20 then prints a boarding ticket describing boarding reservation information and guidance information about procedures up to boarding (step S212) and ends the process.

### [Security Inspection Procedure]

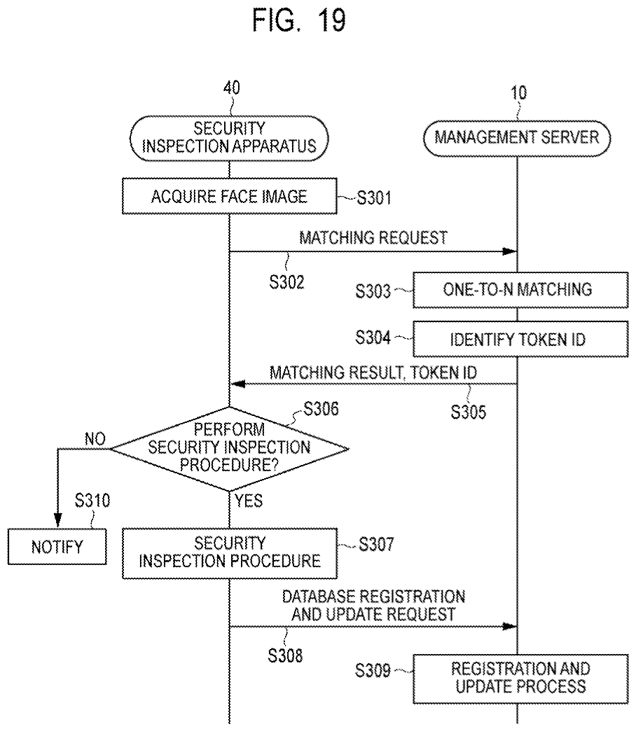

FIG. 19 is a sequence chart illustrating an example of the process during a security inspection procedure of the information processing system Sys according to the present example embodiment. Note that, since each process of identity verification in a baggage drop-off procedure, in a departure inspection procedure, and at a boarding gate is substantially the same as that in FIG. 19, the description thereof will be omitted.

First, in response to detecting a user's face by the biometric information acquisition device 408, the security inspection apparatus 40 captures an image of the user's face to acquire a user face image (step S301).

Next, the security inspection apparatus 40 transmits a matching request including the captured face image of the user to the management server 10 (step S302). In this step, the security inspection apparatus 40 requests the management server 10 to perform one-to-N matching between the captured face image of the user and a plurality of registered face image registered in the token ID information DB 10a of the management server 10.

Next, in response to receiving the captured face image and the matching request from the security inspection apparatus 40, the management server 10 performs the one-to-N matching between the captured face image of the user and the registered face images (step S303).

Next, the management server 10 determines the token ID provided that the matching result in step S303 is that the matching is successful (step S304).

Next, the management server 10 transmits the matching result and the token ID to the security inspection apparatus 40 (step S305). Further, to perform a security inspection procedure, the management server 10 transmits information associated with the registered face image to the security inspection apparatus 40 together with the matching result.

Next, in response to receiving the matching result from the management server 10, the security inspection apparatus 40 determines whether or not a security inspection procedure is ready to be performed on the user (step S306). In this step, if the security inspection apparatus 40 determines that the security inspection procedure on the user is not ready to be performed (step S306: NO), the security inspection apparatus 40 provides a notification of an error message (step S310) and ends the process.

In response, if the security inspection apparatus 40 determines that the security inspection procedure on the user is ready to be performed (step S306: YES), that is, if the matching result is that the matching is successful and the token ID of the user is identified, the security inspection apparatus 40 performs the security inspection procedure (step S307). Next, in response to completion of the security inspection procedure, the security inspection apparatus 40 transmits the database registration and update request to the management server 10 (step S308).

Then, in response to receiving the database registration and update request from the security inspection apparatus 40, the management server 10 performs a registration process and an update process on the passage history information DB 10b and the operation information DB 10c (step S309) and ends the process.

[Guidance about Place of Quarantine Screening]

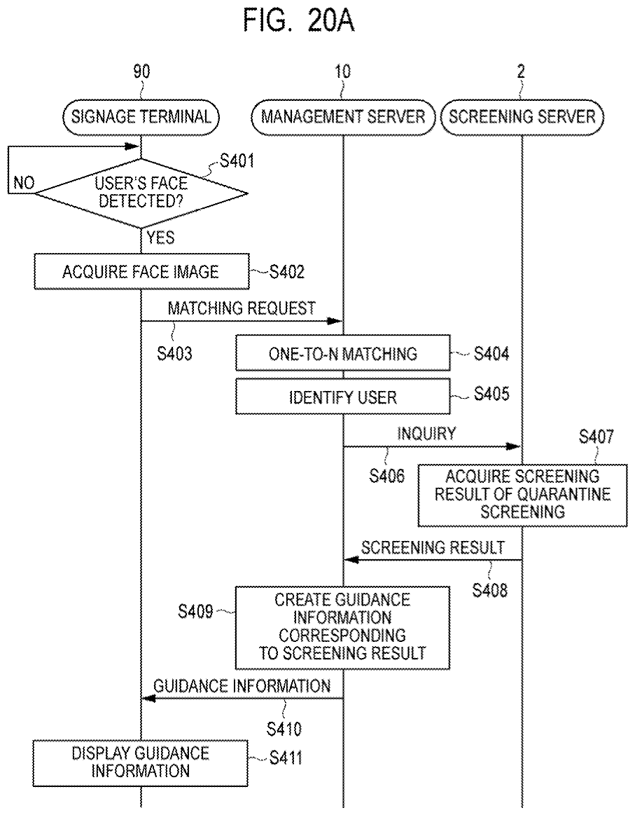

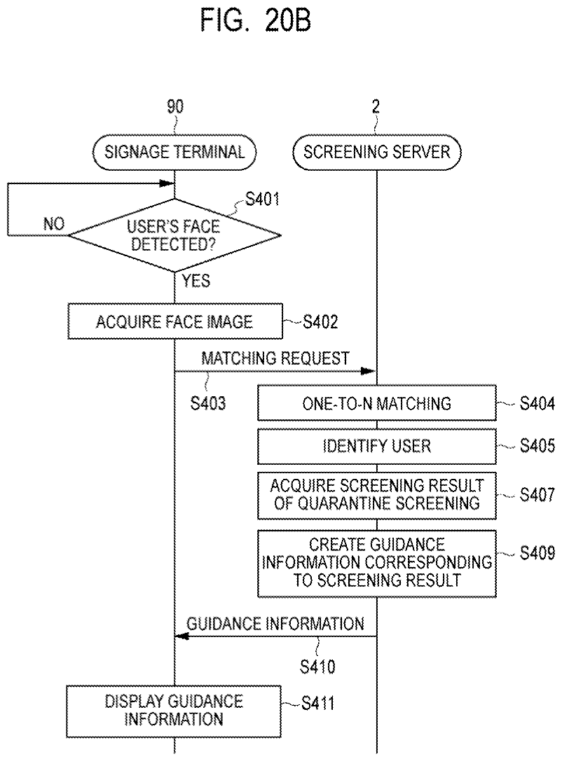

FIG. 20A and FIG. 20B are sequence charts illustrating an example a guidance process in the second country of the information processing system Sys according to the present example embodiment. Firstly, a case where the signage terminal 90 acquires a screening result from the screening server 2 via the management server 10 of an airport system will be described with reference to FIG. 20A.

First, the signage terminal 90 constantly or periodically captures an image of an area in front of the signage terminal 90 by using the biometric information acquisition device 908 and determines whether or not a user's face is detected (step S401).

Next, the signage terminal 90 acquires a face image of the user standing in front of the signage terminal 90 from the captured image (step S402).

Next, the signage terminal 90 transmits a matching request including the user face image to the management server 10 (step S403). Accordingly, the signage terminal 90 requests the management server 10 to perform one-to-N matching between the captured face image of the user and a plurality of registered face images registered in the token ID information DB 10a.

Next, the management server 10 performs the one-to-N matching between the captured face image of the user received from the signage terminal 90 and the registered face images stored in the token ID information DB 10a (step S404).

Next, if the matching result is that the matching is successful, the management server 10 identifies the user (step S405). Specifically, in response to identifying the token ID of the user, the management server 10 identifies user information on the user, such as a passport number, boarding reservation information, a registered face image, or the like, from the operation information DB 10c by using the token ID as a key.

Next, the management server 10 inquires the screening server 2 for the screening result of the quarantine screening based on the user information on the identified user (step S406).

Next, the screening server 2 acquires the screening result from the quarantine screening information DB 2a based on the user information (for example, a face image or a passport number) received from the management server 10 (step S407) and transmits this screening result to the management server 10 (step S408).

Next, the management server 10 creates guidance information corresponding to the screening result received from the screening server 2 (step S409) and then transmits the guidance information to the signage terminal 90 (step S410).

Then, in response to receiving the guidance information from the management server 10, the signage terminal 90 displays the guidance information to the user on the display device 906 (step S411) and ends the process.

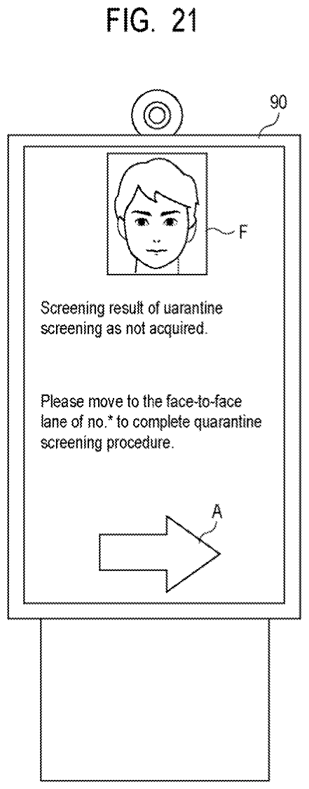

FIG. 21 is a diagram illustrating an example of a guidance window displayed on the signage terminal 90. In this example, a user face image F, a message to guide the user for a procedure place (“Screening result of quarantine screening was not acquired. Please move to the face-to-face lane of No.* to complete quarantine screening procedure.”), and an arrow A indicating the moving direction are displayed in the window.

Next, a case where the signage terminal 90 directly acquires a screening result of quarantine screening from the screening server 2 will be described with reference to FIG. 20B. Note that description for parts common to FIG. 20A will be omitted below, and different features will be described in detail.

In step S403, the signage terminal 90 transmits a matching request including the user face image to the screening server 2. Accordingly, the signage terminal 90 requests the screening server 2 to perform one-to-N matching between the captured face image of the user and a plurality of registered face images registered in the quarantine screening information DB 2a.

In step S404, the screening server 2 performs the one-to-N matching between the captured face image of the user received from the signage terminal 90 and the registered face images stored in the quarantine screening information DB 2a.

Next, if the matching result is that the matching is successful, the screening server 2 identifies the user (step S405). The process then proceeds to step S407.

In step S407, the screening server 2 acquires a screening result of the identified user from the quarantine screening information DB 2a. The process then proceeds to step S409.

In step S409, the screening server 2 creates guidance information corresponding to the screening result. The screening server 2 then transmits the guidance information to the signage terminal 90 (step S410).

Then, in response to receiving the guidance information from the screening server 2, the signage terminal 90 displays the guidance information to the user on the display device 906 (step S411) and ends the process.

In such a way, the information processing system Sys may be a system configuration in which the signage terminal 90 utilizes face authentication to inquire the screening server 2 and directly acquires a screening result of quarantine screening from the screening server 2.

Note that the guidance process illustrated in FIG. 20A, FIG. 20B, and FIG. 21 may be performed by a method other than face authentication. For example, the guidance process may be performed by using the medium reading device 909 of the signage terminal 90 to read a two-dimensional barcode displayed on the user terminal 1 or the like.

### [Quarantine Screening Procedure (1)]

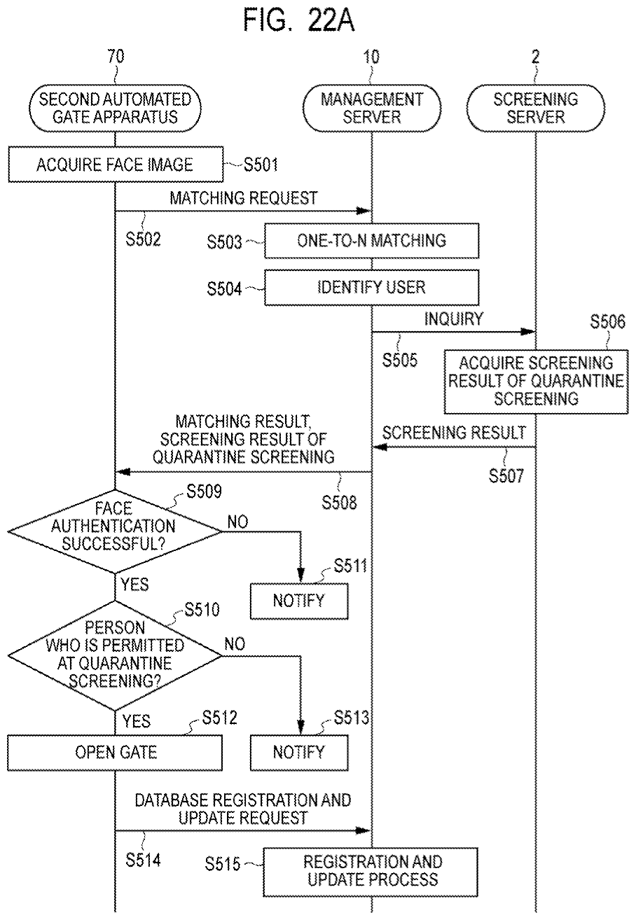

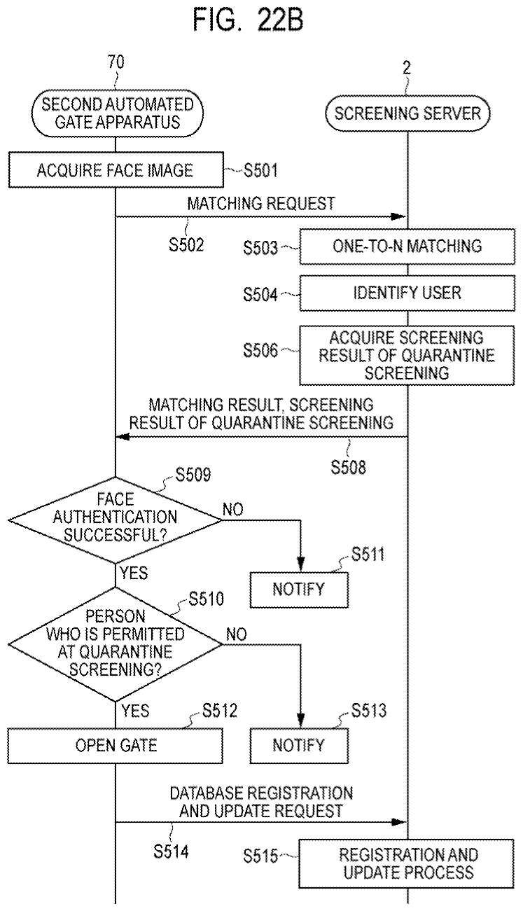

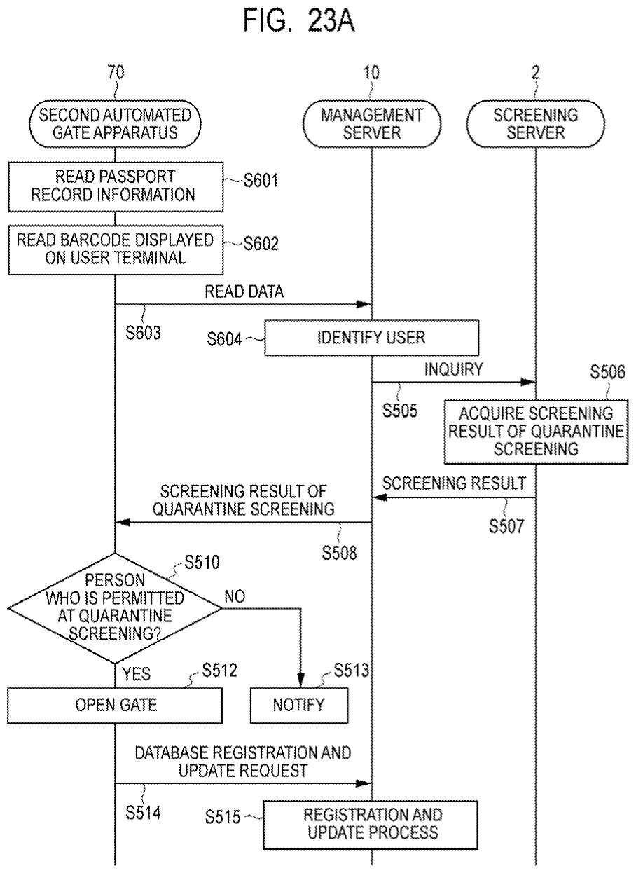

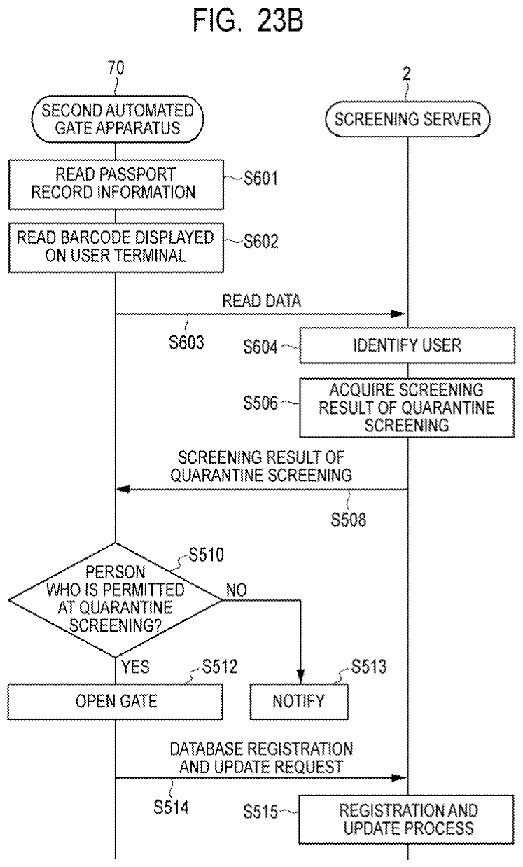

FIG. 22A to FIG. 23B are sequence charts illustrating examples of the process in the quarantine screening procedure of the information processing system Sys according to the present example embodiment. FIG. 22A and FIG. 22B illustrate a process in a face authentication mode. On the other hand, FIG. 23A and FIG. 23B illustrate a process in a barcode authentication mode.

Firstly, a case where the second automated gate apparatus 70 acquires a screening result of quarantine screening from the screening server 2 via the management server 10 of the airport system while utilizing face authentication will be described with reference to FIG. 22A.

First, in response to detecting a user's face by using the biometric information acquisition device 708, the second automated gate apparatus 70 captures an image of the user's face to acquire a user face image (step S501).

Next, the second automated gate apparatus 70 transmits a matching request including the captured face image of the user to the management server 10 (step S502). In this step, the second automated gate apparatus 70 requests the management server 10 to perform one-to-N matching between the captured face image of the user and a plurality of registered face image registered in the token ID information DB 10a of the management server 10.

Next, in response to receiving the captured face image and the matching request from the second automated gate apparatus 70, the management server 10 performs the one-to-N matching between the captured face image of the user and the registered face images (step S503).

The management server 10 identifies the user provided that the matching result in step S503 is that the matching is successful (step S504). Specifically, in response to identifying a token ID of the user, the management server 10 identifies user information on the user, such as a passport number, boarding reservation information, a registered face image, or the like, from the operation information DB 10c by using the token ID as a key.

Next, the management server 10 inquires the screening server 2 for the screening result of the quarantine screening based on the user information on the identified user (step S505).

Next, the screening server 2 acquires the screening result from the quarantine screening information DB 2a based on the user information (for example, a face image or a passport number) received from the management server 10 (step S506) and transmits this screening result to the management server 10 (step S507).

Next, the management server 10 transmits the matching result at the management server 10 and the screening result of quarantine screening acquired from the screening server 2 to the second automated gate apparatus 70 (step S508). Further, the management server 10 transmits information associated with the registered face image of the user in the operation information DB 10c to the second automated gate apparatus 70.

Next, the second automated gate apparatus 70 determines whether or not the face authentication is successful at the management server 10 (step S509). In this step, if the second automated gate apparatus 70 determines that the face authentication failed (step S509: NO), the second automated gate apparatus 70 provides a notification of an error message (step S511) and ends the process.

In contrast, if the second automated gate apparatus 70 determines that the face authentication is successful (step S509: YES), the process proceeds to step S510.

In step S510, the second automated gate apparatus 70 determines whether or not the user is a person who is permitted by the quarantine screening in the second country. In this step, if the second automated gate apparatus 70 determines that the user is a person who is permitted by the quarantine screening (step S510: YES), the process proceeds to step S512.

In contrast, if the second automated gate apparatus 70 determines that the user is not a person who is permitted by the quarantine screening (step S510: NO), the process proceeds to step S513.

In step S512, the second automated gate apparatus 70 opens the gate 710. This enables the user to exit the quarantine station and move to the entry inspection site.

In step S513, the second automated gate apparatus 70 notifies the user of a message to urge the user to have face-to-face quarantine screening and ends the process.

In step S514, the second automated gate apparatus 70 transmits a database registration and update request to the management server 10 in response to completion of the quarantine screening procedure.

Then, in response to receiving the database registration and update request from the second automated gate apparatus 70, the management server 10 performs a registration process and an update process on the passage history information DB 10b and the operation information DB 10c (step S515) and ends the process.

Next, a case where the second automated gate apparatus 70 directly acquires a screening result of quarantine screening from the screening server 2 by using face authentication will be described with reference to FIG. 22B. Note that description for parts common to FIG. 22A will be omitted below, and different features will be described in detail.

In step S502, the second automated gate apparatus 70 transmits the captured face image of the user to the screening server 2. Accordingly, the second automated gate apparatus 70 requests the screening server 2 to perform one-to-N matching between the captured face image of the user and a plurality of registered face images registered in the quarantine screening information DB 2a.

In step S503, in response to receiving the captured face image and the matching request from the second automated gate apparatus 70, the screening server 2 performs the one-to-N matching between the captured face image of the user and the registered face images.

Next, the screening server 2 identifies the user provided that the matching result in step S503 is that the matching is successful (step S504). The process then proceeds to step S506.

In step S506, the screening server 2 acquires a screening result of the identified user from the quarantine screening information DB 2a. The process then proceeds to step S508.

In step S508, the screening server 2 transmits the matching result and the screening result of quarantine screening to the second automated gate apparatus 70. Then, in the second automated gate apparatus 70, the process of step S509 to step S513 is performed.

In step S514, in response to completion of the quarantine screening procedure, the second automated gate apparatus 70 transmits a database registration and update request to the screening server 2.

Then, in response to receiving the database registration and update request from the second automated gate apparatus 70, the screening server 2 performs a registration process and an update process on the quarantine screening information DB 2a (step S515) and ends the process.

In such a way, the information processing system Sys may be a system configuration in which the second automated gate apparatus 70 utilizes face authentication to inquire the screening server 2 and directly acquires a screening result of quarantine screening from the screening server 2.

### [Quarantine Screening Procedure (2)]

Next, the process in the barcode authentication mode will be described with reference to FIG. 23A and FIG. 23B.

Firstly, a case where the second automated gate apparatus 70 acquires a screening result of quarantine screening from the screening server 2 via the management server 10 of the airport system while utilizing barcode authentication will be described with reference to FIG. 23A. Note that description for parts common to FIG. 22A and FIG. 22B will be omitted below, and different features will be described in detail.

First, the second automated gate apparatus 70 uses the medium reading device 709 to read passport record information (step S601). Note that, since identity verification of the user can also be made by using only a barcode, the process (the reading process of a passport) of step S601 is optional and may be omitted.

Next, the second automated gate apparatus 70 uses the medium reading device 709 to read a barcode displayed on the user terminal 1 (step S602). The barcode illustrated in FIG. 17 as an example includes user authentication information and information for identifying a screening result of the user in the screening server 2 (the quarantine screening information DB 2a).

Next, the second automated gate apparatus 70 transmits read data on the passport and the barcode to the management server 10 (step S603).

Next, the management server 10 identifies the user based on the read information on the passport (for example, the passport number) or barcode record information (step S604). The process of step S505 to step S515 is then performed in the same manner as the case of the FIG. 22A (except for step S509 and step S511).

Next, a case where the second automated gate apparatus 70 directly acquires a screening result of quarantine screening from the screening server 2 by using barcode authentication will be described with reference to FIG. 23B. Note that description for parts common to FIG. 22A and FIG. 23A will be omitted below, and different features will be described in detail.

In step S603, the second automated gate apparatus 70 transmits the read data on the passport and the barcode to the screening server 2.

Next, the screening server 2 identifies the user by referencing the quarantine screening information DB 2a based on the read information on the passport (for example, the passport number) or the barcode record information (for example, the user ID, the passport number, the reservation number) (step S604). The process then proceeds to step S506.

In step S506, the screening server 2 acquires the screening result of the identified user from the quarantine screening information DB 2a. The process then proceeds to step S508.

In step S508, the screening server 2 transmits the screening result of quarantine screening to the second automated gate apparatus 70. Then, in the second automated gate apparatus 70, the process of step S510 to step S513 is performed.

In step S514, in response to completion of the quarantine screening procedure, the second automated gate apparatus 70 transmits a database registration and update request to the screening server 2.

Then, in response to receiving the database registration and update request from the second automated gate apparatus 70, the screening server 2 performs a registration process and an update process on the quarantine screening information DB 2a (step S515) and ends the process.

In such a way, the information processing system Sys may be a system configuration in which the second automated gate apparatus 70 utilizes barcode authentication to inquire the screening server 2 and directly acquires a screening result of quarantine screening from the screening server 2.

As described above, according to the present example embodiment, based on health information uploaded from the user terminal 1 to the management server 10, it is possible for a user to complete quarantine screening as the screening related to entry to the second country before the user scheduled to travel from the first country to the second country reaches the departure airport of the first country or before the user performs a procedure related to departure at the departure airport.

Accordingly, since the user is not required to actually undergo the inspection imposed on an entrant to the second country at the departure airport of the first country or a part of the inspection can be omitted, the time spent at the airport can be shortened. Furthermore, according to the present example embodiment, since the health condition of the user can be confirmed on the airport system side, this can contribute to prevention of the spread of infection of contagious diseases.

### Second Example Embodiment

The information processing system Sys in the present example embodiment will be described below. Note that references common to the references provided in the drawings in the first example embodiment represent the same components. Description of the features common to the first example embodiment will be omitted, and different features will be described in detail.

The present example embodiment differs from the first example embodiment in that screening based on entry history information on the user to the second country and screening based on marked person information in the second country are performed in addition to quarantine screening based on the health information on the user as the screening related to entry to the second country by the user. In the present example embodiment, the screening related to entry to the second country is referred to as “pre-immigration inspection (pre-clearance)”. Note that, although screening other than quarantine screening may be performed by a server of a government agency that is different from the government agency which is responsible for the quarantine screening, all types of screening are performed by the same screening server 2 in the present example embodiment.

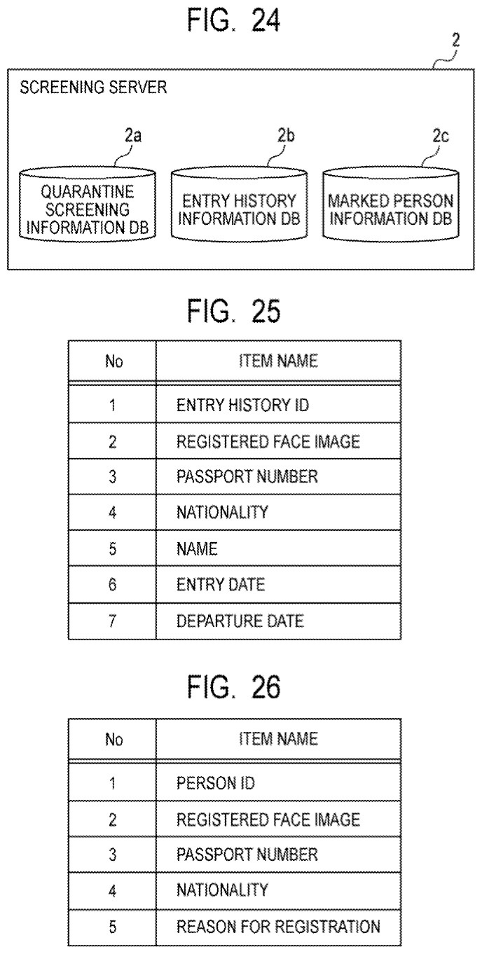

FIG. 24 is a block diagram illustrating an example of the overall configuration of the screening server 2 according to the present example embodiment. As illustrated in FIG. 24, the screening server 2 of the present example embodiment further includes an entry history information DB 2b and a marked person information DB 2c.

The entry history information DB 2b is a database that stores entry history information on all entrants who have entered the second country within a past predetermined period. FIG. 25 is a diagram illustrating an example of information stored in the entry history information DB 2b. The entry history information DB 2b includes data items of an entry history ID, a registered face image, a passport number, a nationality, a name, an entry date, and a departure date.

The marked person information DB 2c is a database that stores information on a person recognized as a marked person in the second county, that is, stores a black list. FIG. 26 is a diagram illustrating an example of information stored in the marked person information DB 2c. The marked person information DB 2c includes data items of a person ID, a registered face image, a passport number, a nationality, and a reason for registration. The person ID is an identifier that uniquely identifies a marked person. Note that the entry history information DB 2b and the marked person information DB 2c may be aggregated as a single database.

### [Pre-Immigration Inspection Procedure]

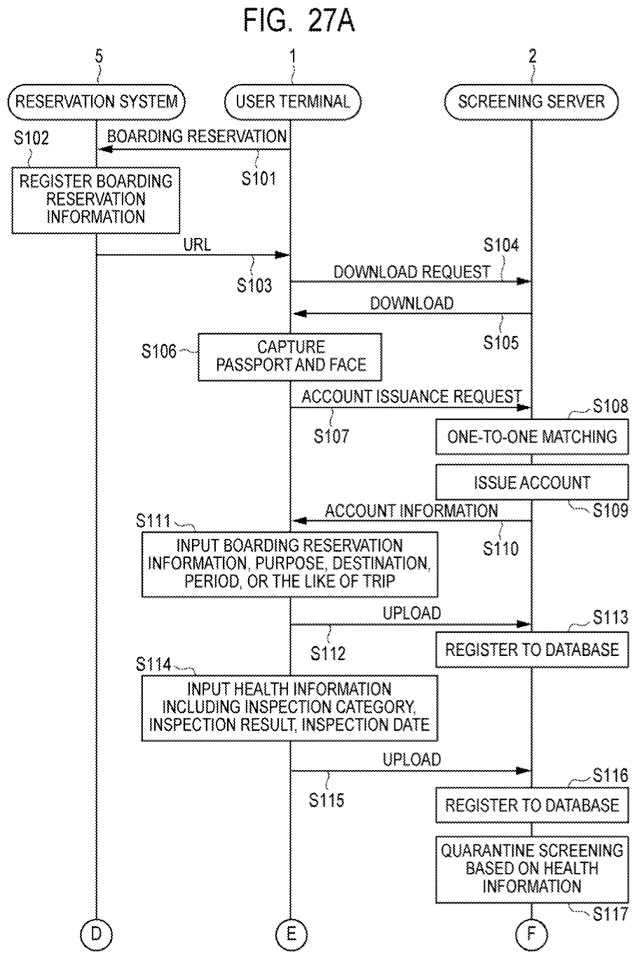

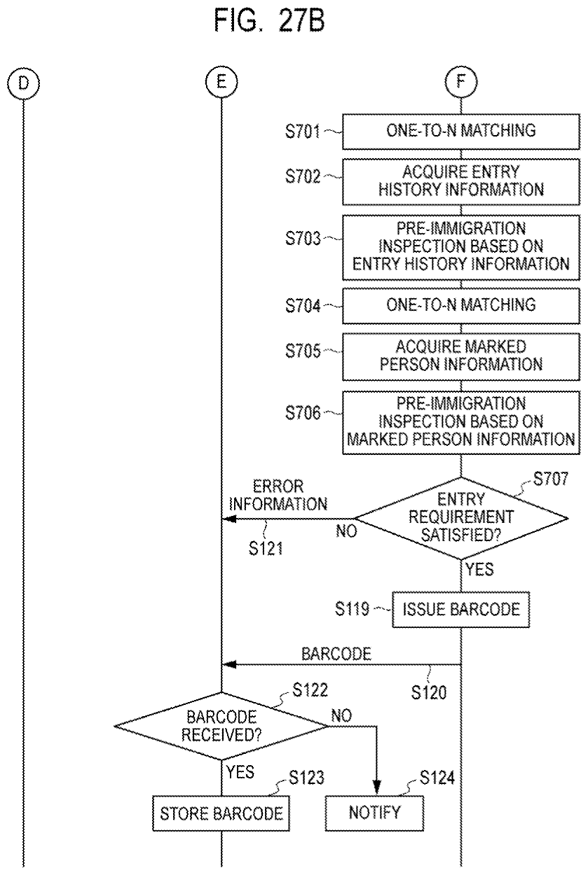

FIG. 27A and FIG. 27B are sequence charts illustrating an example of the process in the pre-immigration inspection procedure to the second country of the information processing system Sys according to the present example embodiment. Since the process in FIG. 27A and FIG. 27B is common to that in FIG. 13A and FIG. 13B of the first example embodiment except for step S701 to step S707, description of the common features will be omitted, and different features will be described in detail.

In response to completion of the quarantine screening based on health information in step S117, the screening server 2 performs one-to-N matching between the registered face image of the user acquired from the quarantine screening information DB 2a and the registered face images of registrants stored in the entry history information DB 2b (step S701).

Next, the screening server 2 acquires entry history information on the user in the second country from the entry history information DB 2b provided that the matching result of the matching process in step S702 is that the matching is successful (step S702).

Next, the screening server 2 performs pre-immigration inspection based on the entry history information (step S703). Specifically, the screening server 2 determines whether or not an entry history to the second country is present for the user.

Next, the screening server 2 performs one-to-N matching between the registered face image of the user acquired from quarantine screening information DB 2a and the registered face images of registrants stored in the marked person information DB 2c (step S704).

Next, the screening server 2 acquires marked person information on the user in the second country from the marked person information DB 2c provided that the matching result of the matching process in step S704 is that the matching is successful (step S705).

Next, the screening server 2 performs pre-immigration inspection based on the marked person information (step S706). Specifically, the screening server 2 determines whether or not the user is a person who has been recognized as a marked person in the second country. Note that the order of performing the quarantine screening (step S117), the pre-immigration inspection based on entry history information (step S703), and pre-immigration inspection based on marked person information (step S706) is not limited to the above. Any of the inspection may be performed earlier, or some of the inspection may be performed in parallel.

Next, the screening server 2 determines whether or not the user satisfies entry requirements for the second country based on all the screening results of step S117, step S703, and step S706 (step S707). In this step, if the screening server 2 determines that the user satisfies the entry requirements for the second country (step S707: YES), the process proceeds to step S119.

In contrast, if the screening server 2 determines that the user does not satisfy the entry requirements for the second country (step S707: NO), the process proceeds to step S121. The process of step S119 and the process on and after the step S121 are the same as those of the first example embodiment.

Note that, in step S707, it can be determined that the entry requirements are satisfied provided that all of (A) to (D) below are satisfied, for example.

(A) The inspection result included in health information is “negative”.
(B) The inspection date is within a predetermined period from a scheduled arrival time at the second country (or a scheduled departure time at the first country).
(C) An entry history to the second country is present within a predetermined period.
(D) The user is not a person who is recognized as a marked person in the second country.

However, the determination method is not limited to the above, and any determination method can be set.

As described above, according to the present example embodiment, the user may undergo pre-immigration inspection by taking into consideration of not only health information on the user acquired in the first country but also entry history information or marked person information in the second country. Accordingly, an advantageous effect of being able to more suitably perform pre-immigration inspection can be achieved in addition to the advantageous effects achieved in the first example embodiment.

### Third Example Embodiment

The information processing system Sys in the present example embodiment will be described below. Note that references common to the references provided in the drawings in the first example embodiment represent the same components. Description of the features common to the first example embodiment will be omitted, and different features will be described in detail.

The present example embodiment differs from the first and second example embodiments in further having a function of determining whether or not to permit boarding on an airplane in the first country based on health information that is used in inspection for entry to the second country.

### [Check-In Procedure]

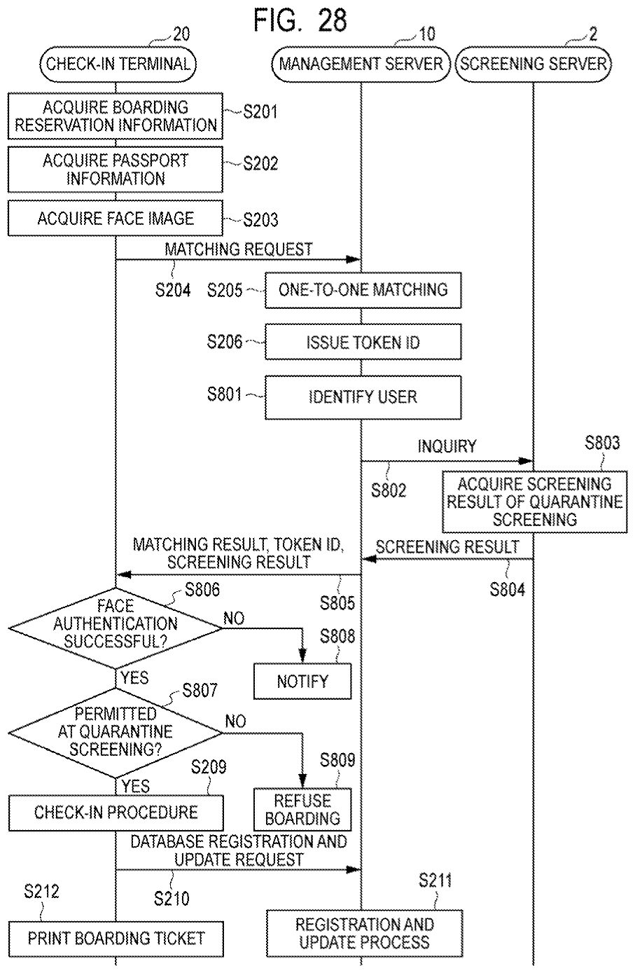

FIG. 28 is a sequence chart illustrating an example of the process in the check-in procedure of the information processing system Sys according to the present example embodiment. Since the process in FIG. 28 is common to that in FIG. 18 of the first example embodiment except for step S801 to step S809, description of the common features will be omitted, and different features will be described in detail.

After issuing a token ID in step S206, the management server 10 identifies the user (step S801). Specifically, the management server 10 identifies user information such as the passport number of the user, the boarding reservation information, the registered face image, or the like.

Next, the management server 10 inquires the screening server 2 for a screening result of quarantine screening based on the user information on the identified user (step S802).

Next, the screening server 2 acquires a screening result from the quarantine screening information DB 2a based on the user information (for example, the passport number) received from the management server 10 (step S803) and transmits this screening result to the management server 10 (step S804).

Next, the management server 10 transmits the matching result in the management server 10, the token ID, and the screening result acquired from the screening server 2 to the check-in terminal 20 (step S805).

Next, the check-in terminal 20 determines whether or not the user face authentication is successful at the management server 10 (step S806). In this step, if the check-in terminal 20 determines that the user face authentication is successful (step S806: YES), the process proceeds to step S807.

In contrast, if the check-in terminal 20 determines that the user face authentication failed (step S806: NO), the check-in terminal 20 provides a notification of an error message (step S808) and ends the process.

In step S807, the check-in terminal 20 determines whether or not the user is a person who has been permitted by quarantine screening in the second country. In this step, if the check-in terminal 20 determines that the user is a person who has been permitted by the quarantine screening (step S807: YES), the process proceeds to step S209. The process on and after step S209 is the same as that of the first example embodiment.

In contrast, if the check-in terminal 20 determines that the user is not a person who has been permitted by the quarantine screening (step S807: NO), the check-in terminal 20 provides a notification of an error message to refuse boarding (step S809), and the process ends.

As described above, according to the present example embodiment, it is possible to determine whether or not to permit each user to board an airplane in the departure airport in the first country based on a result of quarantine screening performed in advance by using health information on the user. Accordingly, an advantageous effect of being able to further prevent the spread of infection in the airport and the airplane can be achieved.

### Fourth Example Embodiment

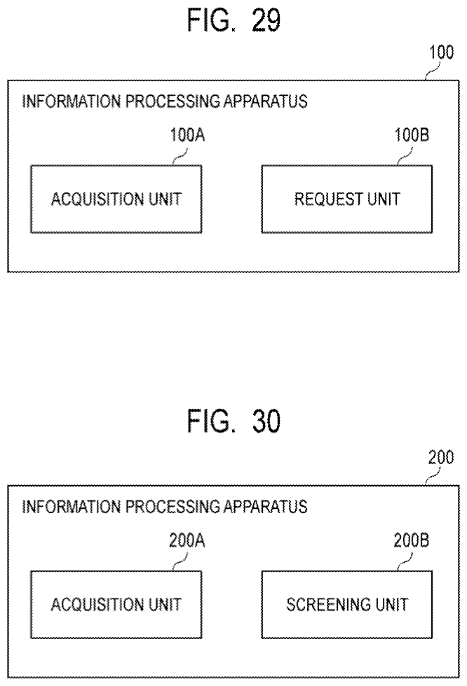

FIG. 29 is a function block diagram of an information processing apparatus 100 according to the present example embodiment. The information processing apparatus 100 includes an acquisition unit 100A and a request unit 100B. The acquisition unit 100A acquires health information about medical treatment performed at a medical institution of the first country on a user scheduled to travel from the first country to the second country. Before the user performs a procedure related to departure, the request unit 100B requests quarantine screening on the user for entry to the second country based on the health information.

According to the present example embodiment, the information processing apparatus 100 that can check the health condition of a user while shortening the time the user spends at an airport can be provided.

### Fifth Example Embodiment

FIG. 30 is a function block diagram of an information processing apparatus 200 according to the present example embodiment. The information processing apparatus 200 includes an acquisition unit 200A and a screening unit 200B. The acquisition unit 200A acquires health information about medical treatment performed at a medical institution of the first country on a user scheduled to travel from the first country to the second country. Before the user performs a procedure related to departure, the screening unit 200B performs quarantine screening on the user for entry to the second country based on the health information.

According to the present example embodiment, the information processing apparatus 200 that can check the health condition of a user while shortening the time the user spends at an airport can be provided.

### Modified Example Embodiment

Although the present invention has been described above with reference to the example embodiments, the present invention is not limited to the example embodiments described above. Various modifications that can be understood by those skilled in the art can be made to the configuration and details of the present invention within the scope not departing from the spirit of the present invention. For example, it should be understood that an example embodiment in which a configuration of a part of any of the example embodiments is added to another example embodiment or an example embodiment in which a configuration of a part of any of the example embodiments is replaced with a configuration of a part of another example embodiment is also an example embodiment to which the present invention may be applied.

In the above example embodiments, the configuration in which, after the user makes a boarding reservation at the reservation system 5 of an airline company by using the user terminal 1, the user terminal 1 receives URL information used for downloading a travel management application from the reservation system 5 has been described. However, such a system that transmits a URL is not limited to only the reservation system 5 of an airline company. For example, after the user makes a reservation of a hotel in the second country from an accommodation reservation system, the accommodation reservation system may transmit the URL to the user terminal 1. Further, requirements for access to the server indicated by the URL may be met without requiring completion of a reservation of a boarding ticket, a hotel, or the like. For example, the user terminal 1 may be used to access, at any timing, the URL indicating an address on the web of the screening server 2 or a system of a company outsourced by the government agency.

Further, in the above example embodiments, the configuration in which the user terminal 1 downloads a travel management application from the screening server 2 based on URL information has been described. However, the system that provides such an application is not limited to the screening server 2. For example, the apparatus such as a server of a company outsourced by a government agency of the first country or the second country, the management server 10 of the first airport system 3, the management server 10 of the second airport system 4, and the like may provide such an application to the user terminal 1 via the network NW.

Further, although the system configuration in which the screening server 2 directly acquires health information from the user terminal 1 has been described in the above example embodiments, the method of acquiring health information at the screening server 2 is not limited thereto. For example, the system configuration may be such that the screening server 2 accesses a database of a medical institution based on input information in the user terminal 1 (for example, a reference number) to acquire health information from the medical institution. In such a case, it is preferable to configure the system to obtain consent from the user about acquisition of particular inspection information from the medical institution.

Further, although the system configuration in which the screening server 2 outputs a barcode only when the screening result of screening in the second country is “permit” has been described in the above example embodiments, the system configuration that may output a barcode even when the screening result is “not permit” may be employed.

Further, although the configuration that performs barcode authentication in a quarantine screening procedure in the second country has been described in the above example embodiments, barcode authentication may be performed at existing touch points in the first country and the second country (security inspection site/departure inspection site/boarding gate/customs inspection site or the like). The same applies to a case where a touch point for checking the health condition of the user is newly provided in the future.

For example, in a quarantine station of the second country, since registered health information can be acquired by face authentication and barcode authentication, no additional inspection is required, and this can shorten the time spent at the airport A2 of the second country. Further, even when additional inspection is performed, since the inspection result in the first country can be shared, safe inspection can be performed.

Although the case where the second automated gate apparatus 70 is installed in the quarantine station has been described in the above example embodiments, the installation place of the second automated gate apparatus 70 is not limited thereto. For example, the second automated gate apparatus 70 may be installed at a touch point newly provided for checking the health condition of the user, on a moving path from an arrival gate to an entry inspection site, or the like.

Further, although the case where an inspection date and an inspection result of inspection provided by a medical institution of the first country are utilized as health information has been described in the above example embodiments, the health information is not limited thereto. For example, a record of vaccination administered, information on the effective period of an administered vaccine, an inspection record of an antibody test and an antigen test, a history of hospital visits, a medication record, a result of physical checkup from a wearable terminal, information on a previous disease, or the like can also be utilized as the health information.

Further, when the health information is an effective period of a vaccine, the screening server 2 can output a screening result of “permit” in quarantine screening if the effective period of the vaccine is effective at the date of entry to the second country or the date of stay in the second country. A case where the effective period of a vaccine is set to six months will be specifically described below as an example. If a scheduled arrival date at the second country (or a scheduled departure date at the first country) is within the effective period, that is, if a scheduled arrival date at the second country (or a scheduled departure date at the first country) is within six months from a vaccination, the screening server 2 can output a screening result of “permit” in quarantine screening.

Subsequently, a case where the stay period is 10 days will be specifically described as an example. If a date of a scheduled arrival date at the second country plus 10 days, that is, a date of departure from the second country is within the effective period, that is, if a date of a scheduled arrival date at the second country plus 10 days is within 6 months from a vaccination, the screening server 2 can output a screening result of “permit” in quarantine screening.

Further, when an application that centrally manages health information on the user in cooperation with a server of a medical institution is installed in the user terminal 1, an inspection result of inspection that the user undergoes at the medical institution may be directly provided from the server of the medical institution to the user terminal 1. Accordingly, the user may utilize the health information at various procedures related to entry to or departure from the country at an airport by starting the application of the user terminal 1 instead of presenting the barcode described above.

Further, in the third example embodiment described above, the configuration in which the management server 10 acquires a screening result of quarantine screening in cooperation for data with the screening server 2 and determines whether or not to permit boarding when the user performs a check-in procedure has been described. However, the touch point where a screening result of quarantine screening can be utilized is not limited to the above. That is, also in a procedure other than the check-in procedure (the baggage drop-off procedure, the security inspection procedure, the departure inspection, identity verification at a boarding gate), a screening result of quarantine screening can be utilized in the same manner as in the check-in procedure.

Furthermore, the health information registered in the information processing system Sys in the example embodiment described above can be utilized at a place other than an airport. For example, when a server of a business entity for hotels or rental cars of the second country is made connectable to the information processing system Sys described above, this enables the user to easily perform a procedure related to stay at a hotel or rental of a car by utilizing barcode authentication and face authentication in a town.

The scope of each of the example embodiments also includes a processing method that stores, in a storage medium, a program that causes the configuration of each of the example embodiments to operate so as to implement the function of each of the example embodiments described above, reads the program stored in the storage medium as a code, and executes the program in a computer. That is, the scope of each of the example embodiments also includes a computer readable storage medium. Further, each of the example embodiments includes not only the storage medium in which the program described above is stored but also the individual program itself.

As the storage medium, for example, a floppy (registered trademark) disk, a hard disk, an optical disk, a magneto-optical disk, a CD-ROM, a magnetic tape, a nonvolatile memory card, or the like can be used. Further, the scope of each of the example embodiments also includes an example that operates on OS to perform a process in cooperation with another software or a function of an add-in board without being limited to an example that performs a process by an individual program stored in the storage medium.

The whole or part of the example embodiments disclosed above can be described as, but not limited to, the following supplementary notes.

### (Supplementary Note 1)

An information processing apparatus comprising:
an acquisition unit that acquires health information about medical treatment performed at a medical institution of a first country on a user scheduled to travel from the first country to a second country; and
a request unit that, before the user performs a procedure related to departure, requests quarantine screening on the user for entry to the second country based on the health information.

### (Supplementary Note 2)

The information processing apparatus according to supplementary note 1,
wherein the acquisition unit further acquires a purpose, a destination, and a period of a trip made by the user for the entry to the second country, and
wherein the request unit requests the quarantine screening including the purpose, the destination, and the period of the trip.

### (Supplementary Note 3)

The information processing apparatus according to supplementary note 1 or 2,
wherein the acquisition unit further acquires a captured face image in which an image of a face of the user is captured and a passport face image recorded on a passport, and
wherein the request unit requests the quarantine screening including the captured face image and the passport face image.

### (Supplementary Note 4)

The information processing apparatus according to supplementary note 3, wherein the acquisition unit acquires the passport face image by capturing an image of a face photograph attached to the passport.

### (Supplementary Note 5)

The information processing apparatus according to supplementary note 3, wherein the acquisition unit acquires the passport face image by reading via wireless communication from a storage medium embedded in the passport.

### (Supplementary Note 6)

The information processing apparatus according to any one of supplementary notes 1 to 5, wherein the acquisition unit acquires an image that identifies a screening result of the quarantine screening from an apparatus that performs the quarantine screening.

### (Supplementary Note 7)

The information processing apparatus according to supplementary note 6, wherein the image is a barcode.

### (Supplementary Note 8)

The information processing apparatus according to any one of supplementary notes 1 to 7, wherein the health information includes an inspection result of inspection imposed on an entrant to the second country and related to a contagious disease.

### (Supplementary Note 9)

The information processing apparatus according to any one of supplementary notes 1 to 8, wherein the health information includes at least one piece of information of a genetic test, a vaccination, an antibody test, and an antigen test taken by the user.

### (Supplementary Note 10)

An information processing apparatus comprising:
an acquisition unit that acquires health information about medical treatment performed at a medical institution of a first country on a user scheduled to travel from the first country to a second country; and
a screening unit that, before the user performs a procedure related to departure, performs quarantine screening on the user for entry to the second country based on the health information.

### (Supplementary Note 11)

The information processing apparatus according to supplementary note 10,
wherein the acquisition unit further acquires a purpose, a destination, and a period of a trip made by the user for the entry to the second country, and
wherein the screening unit performs the quarantine screening including the purpose, the destination, and the period of the trip.

### (Supplementary Note 12)

The information processing apparatus according to supplementary note 10 or 11,
wherein the acquisition unit further acquires entry history information on the user for entry to the second country, and
wherein the screening unit further performs pre-immigration inspection to the second country on the user based on the entry history information.

### (Supplementary Note 13)

The information processing apparatus according to any one of supplementary notes 10 to 12, wherein the acquisition unit acquires the health information from a user terminal possessed by the user.

### (Supplementary Note 14)

The information processing apparatus according to any one of supplementary notes 10 to 12, wherein the acquisition unit acquires the health information from a database of the medical institution based on input information on a user terminal possessed by the user.

### (Supplementary Note 15)

The information processing apparatus according to supplementary note 13 or 14, wherein the screening unit outputs, to the user terminal, an image that identifies a screening result of the quarantine screening.

### (Supplementary Note 16)

The information processing apparatus according to supplementary note 15, wherein the image is a barcode.

### (Supplementary Note 17)

The information processing apparatus according to any one of supplementary notes 13 to 16 further comprising:
a matching unit that performs a matching process between a captured face image of a face of the user captured by the user terminal and a passport face image acquired from a passport by the user terminal; and
a processing unit that, when the user is authenticated by the matching process to be the same person as a person to which the passport was issued, associates the health information and a screening result of the quarantine screening with identification information on the user.

### (Supplementary Note 18)

The information processing apparatus according to supplementary note 17, wherein the processing unit further associates the captured face image or the passport face image as a registered face image with the identification information.

### (Supplementary Note 19)

The information processing apparatus according to any one of supplementary notes 10 to 18, wherein the health information includes an inspection result of inspection imposed on an entrant to the second country and related to a contagious disease.

### (Supplementary Note 20)

The information processing apparatus according to supplementary note 19, wherein when an inspection date of the inspection included in the health information is within a predetermined period from a scheduled departure time at the first country and the inspection result is negative, the screening unit outputs a screening result indicating that a requirement related to quarantine in the second country is satisfied.

### (Supplementary Note 21)

The information processing apparatus according to supplementary note 19, wherein when an inspection date of the inspection included in the health information is within a predetermined period from a scheduled arrival time at the second country and the inspection result is negative, the screening unit outputs a screening result indicating that a requirement related to quarantine in the second country is satisfied.

### (Supplementary Note 22)

The information processing apparatus according to any one of supplementary notes 10 to 21, wherein the health information includes at least one piece of information of a genetic test, a vaccination, an antibody test, and an antigen test taken by the user.

### (Supplementary Note 23)

An information processing system comprising:
the information processing apparatus according to any one of supplementary notes 10 to 22; and
a server,
wherein the server comprises
an identifying unit that identifies the user through another matching process to match another captured face image of the user captured by a face authentication terminal, which performs a procedure operation related to departure in a departure airport of the first country, with a face image registered in advance for the user,
an inquiry unit that inquires an information processing apparatus for a result of the quarantine screening based on identification information on the identified user, and
a determination unit that determines whether or not to permit the user to board an airplane based on the result of the quarantine screening returned in response to the inquiry.

### (Supplementary Note 24)

An information processing system comprising:
the information processing apparatus according to any one of supplementary notes 10 to 22; and
a server,
wherein the server comprises
an identifying unit that identifies the user through another matching process to match another captured face image of the user captured by a display terminal installed in an arrival airport of the second country with a face image registered in advance for the user,
an inquiry unit that inquires an information processing apparatus for a result of the quarantine screening on the user for entry to the second country based on identification information on the identified user, and
a guidance unit that, based on the result of the quarantine screening returned in response to the inquiry, presents guidance information about a place of entry inspection on the display terminal.

### (Supplementary Note 25)

An information processing method comprising:
acquiring health information about medical treatment performed at a medical institution of a first country on a user scheduled to travel from the first country to a second country; and
before the user performs a procedure related to departure, requesting quarantine screening on the user for entry to the second country based on the health information.

### (Supplementary Note 26)

A storage medium storing a program that causes a computer to perform:
acquiring health information about medical treatment performed at a medical institution of a first country on a user scheduled to travel from the first country to a second country; and
before the user performs a procedure related to departure, requesting quarantine screening on the user for entry to the second country based on the health information.

### (Supplementary Note 27)

An information processing method comprising:
acquiring health information about medical treatment performed at a medical institution of a first country on a user scheduled to travel from the first country to a second country; and
before the user performs a procedure related to departure, performing quarantine screening on the user for entry to the second country based on the health information.

### (Supplementary Note 28)

A storage medium storing a program that causes a computer to perform:
acquiring health information about medical treatment performed at a medical institution of a first country on a user scheduled to travel from the first country to a second country; and
before the user performs a procedure related to departure, performing quarantine screening on the user for entry to the second country based on the health information.

### REFERENCE SIGNS LIST

NW network
Sys information processing system
1 user terminal
2 screening server
2a quarantine screening information DB
2b travel history information DB
2c marked person information DB
3 first airport system
4 second airport system
5 reservation system
10 management server
10a token ID information DB
10b passage history information DB
10c operation information DB
20 check-in terminal
30 automatic baggage drop-off machine
40 security inspection apparatus
50 first automated gate apparatus
60 boarding gate apparatus
70 second automated gate apparatus
80 automatic customs gate apparatus
90 signage terminal
100 information processing apparatus
100A acquisition unit
100B request unit
200 information processing apparatus
200A acquisition unit
200B screening unit

What is claimed is:

1. An information processing apparatus comprising:
at least one memory storing instructions; and
at least one processor configured to execute the instructions to:
acquire health information about medical treatment performed at a medical institution of a first country on a user scheduled to travel from the first country to a second country; and
before the user performs a procedure related to departure, request quarantine screening on the user for entry to the second country based on the health information.

2. The information processing apparatus according to claim 1, wherein the at least one processor is further configured to execute the instructions to:
acquire a purpose, a destination, and a period of a trip made by the user for the entry to the second country, and
request the quarantine screening including the purpose, the destination, and the period of the trip.

3. The information processing apparatus according to claim 1, wherein the at least one processor is further configured to execute the instructions to:
acquire a captured face image in which an image of a face of the user is captured and a passport face image recorded on a passport, and
request the quarantine screening including the captured face image and the passport face image.

4. The information processing apparatus according to claim 3, wherein the at least one processor is further configured to execute the instructions to:
acquire the passport face image by capturing an image of a face photograph attached to the passport.

5. The information processing apparatus according to claim 3, wherein the at least one processor is further configured to execute the instructions to:
acquire the passport face image by reading via wireless communication from a storage medium embedded in the passport.

6. The information processing apparatus according to claim 1, wherein the at least one processor is further configured to execute the instructions to:
acquire an image that identifies a screening result of the quarantine screening from an apparatus that performs the quarantine screening.

7. The information processing apparatus according to claim 6, wherein the image is a barcode.

8. The information processing apparatus according to claim 1, wherein the health information includes an inspection result of inspection imposed on an entrant to the second country and related to a contagious disease.

9. The information processing apparatus according to claim 1, wherein the health information includes at least one piece of information of a genetic test, a vaccination, an antibody test, and an antigen test taken by the user.

10.-24. (canceled)

25. An information processing method comprising:
acquiring health information about medical treatment performed at a medical institution of a first country on a user scheduled to travel from the first country to a second country; and
before the user performs a procedure related to departure, requesting quarantine screening on the user for entry to the second country based on the health information.

26. A non-transitory storage medium storing a program that causes a computer to perform:
acquiring health information about medical treatment performed at a medical institution of a first country on a user scheduled to travel from the first country to a second country; and
before the user performs a procedure related to departure, requesting quarantine screening on the user for entry to the second country based on the health information.

27.-28. (canceled)
